(19) United States
(12) Patent Application Publication
(10) Pub. No.: US 2021/0247370 A1
(43) Pub. Date: Aug. 12, 2021

(54) DEVICES AND METHODS TO COMBINE NEURONS WITH SILICON DEVICES

(71) Applicant: KONIKU INC., San Rafael, CA (US)

(72) Inventors: Oshiorenoya E. AGABI, Dublin, CA (US); Renaud RENAULT, Fremont, CA (US); Winston MANN, Fremont, CA (US); Jean-Charles NEEL, San Francisco, CA (US); Benjamin SADRIAN, Union City, CA (US); Yunchao GAI, Newark, CA (US)

(21) Appl. No.: 17/270,846

(22) PCT Filed: Aug. 24, 2018
(86) PCT No.: PCT/US18/48012

§ 371 Date: Feb. 23, 2021

Related U.S. Application Data
(60) Provisional application No. 62/549,675, filed on Aug. 24, 2017.
(60) Provisional application No. 62/717,284, filed on Aug. 10, 2018.

(51) Int. Cl. G01N 33/00; G01N 1/22; G01N 1/24

(52) U.S. Cl. CPC G01N 33/0057; G01N 1/2273; G01N 2001/245; G01N 1/24; G01N 2001/2276; G01N 1/2205

(57) ABSTRACT

A device housing that is designed to maximize the dwell time for volatile compounds drawn into a cell-based compound detection device is described. The modular functional components of the cell-based detection devices are also described.

### CROSS-REFERENCE

This application claims the benefit of U.S. Provisional Application No. 62/549,675, filed on Aug. 24, 2017, and of U.S. Provisional Application No. 62/717,284, filed on Aug. 10, 2018, both of which applications are incorporated herein by reference.

### BACKGROUND

Cell-based sensor devices for detection of volatile compounds in air samples or solubilized compounds in liquid samples may have important applications in a variety of industries

### SUMMARY

Disclosed herein are device housings comprising: a) a shell component, wherein the shell component comprises: i) a structure comprising a sigmoidal shape that is rotationally symmetric about a single axis; ii) two or more air inlets positioned concentrically around the single axis; iii) two or more air outlets positioned concentrically around the single axis; and b) a baseplate component; wherein the sigmoidal shape of the shell component and positions of the two or more air inlets and two or more air outlets are configured to prolong a dwell time of molecules or particles transported into an interior of the device housing by a flow of air.

In some embodiments, the baseplate component further comprises an attachment structure. In some embodiments, the attachment structure comprises a permanent adhesive, a non-permanent adhesive, a Velcro component, a magnetic component, a hook, a wearable attachment, or any combination thereof. In some embodiments, the shell component is an injection-molded or three-dimensional printed part. In some embodiments, the shell component is fabricated from a polymer, a glass, a metal, a ceramic, or any combination thereof.

Also disclosed herein are devices for detection of compounds, the device comprising: a) a device housing; b) a microfluidics layer comprising a fluid inlet, a fluid outlet, one or more fluid chambers, and a semipermeable membrane configured to promote gas exchange between air within the device housing and the one or more fluid chambers, wherein the one or more fluid chambers are configured to support neurons that have been genetically-engineered to express one or more odorant receptors; c) a structured microelectrode array (MEA) comprising a plurality of electrodes configured to provide electrical stimuli to, or record electrical signals generated by, the neurons in the one or more fluid chambers.

In some embodiments, the device housing comprises: a) a shell component, wherein the shell component comprises: i) a structure comprising a sigmoidal shape that is rotationally symmetric about a single axis; ii) two or more air inlets positioned concentrically around the single axis; iii) two or more air outlets positioned concentrically around the single axis; and b) a baseplate component; wherein the sigmoidal shape of the shell component and positions of the two or more air inlets and two or more air outlets are configured to prolong a dwell time of compounds transported into an interior of the device housing by a flow of air.

In some embodiments, the device further comprises a pre-concentrator module configured to concentrate compounds from air and maximize a dwell time of the compounds at a surface of the semi-permeable membrane. In some embodiments, the pre-concentrator module comprises: a) a fan configured to draw air into the device; b) a high efficiency particulate absorber (HEPA) filter configured to remove contaminant particles from the air drawn into the device; and c) an air director configured to concentrate and direct the flow of air towards the surface of the semi-permeable membrane. In some embodiments, the neurons have been genetically-engineered to respond to photo-stimulation. In some embodiments, the device further comprises a light-emitting diode (LED) array configured to stimulate the neurons in the one or more fluid chambers. In some embodiments, the device further comprises growth medium and waste cartridges so that the device is self-contained and configured to function without maintenance for a specified period of time. In some embodiments, the device is configured to function without maintenance for at least 1 week. In some embodiments, the device is configured to function without maintenance for at least 1 month. In some embodiments, the device is configured to function without maintenance for at least 3 months. In some embodiments, the device further comprises a field programmable gate array (FPGA) or processor configured to perform signal processing of electrical signals recorded by the electrodes of the MEA. In some embodiments, the device further comprises a field programmable gate array (FPGA) or processor configured to perform electrical stimulation of the neurons in the one or more fluid chambers using the electrodes of the MEA. In some embodiments, the device further comprises a field programmable gate array (FPGA) or processor configured to activate one or more LEDs of the LED array to stimulate the neurons in the one or more fluid chambers, and to perform signal processing of electrical signals recorded by the electrodes of the MEA, thereby providing a test of neuron response. In some embodiments, the baseplate component comprises an attachment structure configured to attach the device to an internal or external wall, an internal or external floor, a ceiling of a room, or a roof of a building. In some embodiments, the baseplate component comprises an attachment structure configured to attach the device to a bicycle, motorcycle, automobile, plane, helicopter, robot, drone, or other manned or unmanned aerial vehicle. In some embodiments, the baseplate component comprises an attachment structure configured to permit the device to be worn by an animal or a human.

Disclosed herein are systems comprising: a) a chamber positioned within a space, wherein the chamber comprises a cell expressing one or more cell-surface receptors, and wherein, when a binding event occurs between one or more of the one or more cell-surface receptors and a compound present within the space an electrical signal results in response to the binding event; b) at least one electrode positioned within the chamber and configured to measure the electrical signal that results in response to the binding event; and c) a controller configured to receive the electrical signal and compute a presence or absence of the compound within the space.

In some embodiments, the compound comprises a volatile compound. In some embodiments, the cell is a neuron. In some embodiments, the cell is modified to express one or more cell-surface receptors. In some embodiments, the one or more cell-surface receptors comprise an odorant receptor. In some embodiments, the cell is genetically modified to express the one or more cell-surface receptors. In some embodiments, the electrical signal comprises an action potential, a cell membrane depolarization, or a combination thereof. In some embodiments, the space is a public space. In some embodiments, the space is an airport, a train station, a bus station, a sports arena, a performing arts center, a school, a medical facility, or any combination thereof In some embodiments, the space is a residential setting.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-B illustrate two approaches to maintaining the security of a space.

FIGS. 2A-B illustrate the chemical or odor wake associated with individuals as they pass through a space such as an airport or other public space.

FIG. 3 provides an illustration of a microelectrode array (MEA) that may be incorporated in the disclosed devices.

FIG. 4 provides a non-limiting example of a microfluidics layer design for the disclosed devices.

FIG. 5 provides a non-limiting illustration of a fan designed to be part of a pre-concentrator module that may be incorporated in some embodiments of the disclosed devices.

FIG. 6 provides a non-limiting example of a HEPA filter design that may be incorporated in some embodiments of the disclosed devices.

FIG. 7 provides a top view of an air director that may be incorporated in some embodiments of the disclosed devices.

FIG. 8 provides a bottom view of an air director that may be incorporated in some embodiments of the disclosed devices.

FIG. 9 provides a non-limiting example of a semi-permeable membrane that may be incorporated in some embodiments of the disclosed devices.

FIG. 10 provides a non-limiting example of a modular assembly comprising a semi-permeable membrane, microfluidics layer, three-dimensional structured microelectrode array (3D-SMEA), and a mounting frame.

FIG. 11 provides a non-limiting example of a light-emitting diode (LED) array that may be incorporated in some embodiments of the disclosed devices.

FIG. 12 provides a non-limiting example of food (i.e., growth medium) and waste cartridges that may be incorporated in some embodiments of the disclosed devices.

FIG. 13 provides a non-limiting example of a modular assembly comprising a field programmable gate array (FPGA) and mounting frame.

FIG. 14 provides a non-limiting example of a modular battery pack assembly that may be incorporated into the disclosed devices.

FIGS. 15A-B provide non-limiting examples of an assembled device (shown without the housing).

FIG. 16 is a top view of the device showing style, form or components of the device.

FIG. 17 is a side view of the device showing a curvature which may promote an aerodynamically efficient flow of air from one or more top concentric slits to one or more secondary air inlets.

FIG. 18 is a bottom view of the device showing an attachment structure which may comprise an adhesive (such as Velcro), a magnetic component, a hook, a wearable attachment or any combination thereof that may associate to a surface, a host or facilitating embodiment in a secondary system.

FIG. 19 is a side-oblique view of the device which shows the underside openings that may facilitate airflow and that may blend with the secondary air flow views and shows the characteristic top slits.

FIG. 20 is an oblique-bottom view of the device which shows an example attachment structure (such as a circular attachment). One or more electronic ports may connect to a central wired bus on the back end—such as a device that may be used in tandem to detect a range or panel of molecules or may be used for device redundancy.

FIG. 21 is an oblique top view of the device which shows an interconnectedness of one or more surrounding holes.

FIG. 22 is a side view detailing an elliptical channel which may run around the entire device and a clearer view of a hole which may lead to a central chamber.

FIG. 23 provides a non-limiting example of an assembled device that comprises a HEPA filter bound to a surface of the housing.

FIG. 24 provides a cut-away view of the interior of an assembled device showing the placement of the modular device components in one embodiment.

FIG. 25 provides an example of simulation data for the computed air velocities and wake zones within an air-sampling compound detection device.

FIG. 26 provides an example of simulation data for the computed air velocities near the air outlets of the device that indicate laminar flow of the exiting air.

FIG. 27 provides an example of computational fluid dynamic simulation data for air pressure within an air-sampling compound detection device.

FIG. 28 provides a non-limiting example of experimental data for the detection of explosive compounds using prototypes of the disclosed devices.

### DETAILED DESCRIPTION

Disclosed herein are devices and systems for compound detection that comprise neurons that have been genetically-engineered to express odorant receptors or other cell surface receptors, as well as device housing designs and modular functional components that provide enhanced flexibility in device configuration so that device performance may be optimized for a variety of different detection applications. The compound detection capabilities of a given device are determined by the binding specificity of the one or more types of cell-surface receptors expressed in the neurons or other cells incorporated into the device, and may be tailored for a specific application by selecting different types of cells or neurons, and by modifying the receptor proteins expressed therein. In the presence of a sample comprising the compound or analyte of interest, binding of the compound to a cell-surface receptor induces an electrical signal, e.g., a change in transmembrane potential or the induction of an action potential, that may be recorded by an electrode that is in contact with or in close proximity to one or more neurons or cells. The plurality of electrodes in a microelectrode array (MEA) chip, which interfaces with a microfluidics layer within which the population of neurons or other cells resides, allows simultaneous and/or sequential recording of electrical signals produced by a plurality of neurons or other cells within the device. In some instances, the processing of the electrical signals recorded by the electrodes of the MEA chip allows for the detection of the presence of a single compound in a sample. In some instances, the processing of the electrical signals recorded by the electrodes of the MEA chip allows for the detection of the presence of multiple compounds in a sample, and the identification of those compounds. In some instances, the disclosed devices and systems provide a qualitative result for the detection and identification of one or more compounds present in the sample. In some instances, the disclosed devices and systems provide a quantitative result for the detection and identification of multiple compounds present in the sample. In some instances, one or more electrodes of the MEA may also be used to provide an electrical stimulus to one or more neurons or cells within the device as well as to record electrical signals.

As noted, the functional core of the disclosed cell-based detection devices comprises a microelectrode array (MEA), a microfluidics layer, and neurons that have been genetically-engineered to express one or more odorant receptors or other cell surface receptors. Additional functional components may be swapped in or out of the device configuration depending on the application area or industry vertical of interest. Examples of such modular, functional components include, but are not limited to, aerodynamically designed device housings for maximizing the dwell time of volatile compounds within the detection device, pre-concentrator modules for concentrating volatile compounds, fans for drawing air into the device and/or through a pre-concentrator module, high-efficiency particulate absorber (HEPA) filters to exclude airborne particles or contaminants that are not relevant to the detection application at hand, air director components for maximizing the volume of air sampled by the device per unit time (in some embodiments, a pre-concentrator module may comprise one or more fans, HEPA filters, and/or air directors), semi-permeable membranes that facilitate gas exchange between air samples and the culture medium bathing the neurons for air sampling applications, liquid sampling interfaces for liquid sampling applications, temperature control units, an optical stimulation system (e.g., a light emitting diode (LED) array) for stimulating neurons that have been modified to respond to photo-stimulation as well as chemical or electrical stimulation (e.g., for monitoring device performance), growth medium (food) and waste cartridges for providing nutrients to and storing waste generated by the neurons in self-contained devices, field programmable gate arrays (FPGAs) or processors for performing pre-processing and/or processing of electrical signals recorded by the microelectrode array or for implementing calibration or performance test algorithms, batteries for providing power to device electronics in self-contained devices, etc.

Examples of applications for the disclosed devices and systems include, but are not limited to, detection of volatile compounds (e.g., explosives or markers for explosives) for public space or private security applications, detection of volatile and/or solubilized compounds in air and/or fluid samples for clinical diagnostics and public health applications, monitoring the degree of ripeness or spoilage of produce or other products in the agricultural industry, and the like. The selection of specific modular functional components for inclusion in specific device configurations may be tailored to the specific application. For example, many applications in the security industry vertical may require the detection of volatile organic compounds (VOCs) in the air. For some applications, the detection of VOCs in air may require a pre-concentrator for collecting a large quantity of air sample. Security applications may also require fast collection and processing of air samples from enclosed or open spaces. They may require the use of high efficiency particulate absorber (HEPA) filters to exclude airborne particles or contaminants that are not relevant to the detection application at hand, as well as a semi-permeable membrane that, as mentioned above, enables gas exchange between the air sample and the neurons that reside within a fluid growth medium. In some security applications, ancillary parts of the device, e.g., the device housing, may be aerodynamically optimized for detection speed and sensitivity.

In another example, deploying the disclosed cell-based detection devices for detection applications that require sensing in fluid samples may require a fluid sample collection system, e.g., a sample collection system which directly transports the fluids to the genetically-engineered neurons for detection of compounds dissolved in the fluid. Such as system may comprise exposing the sensing neurons to a continuous flow of liquid that has been diverted from a main fluid path (e.g., a river or stream in the case of environmental monitoring applications, or a water pipe or effluent pipe in the case of a manufacturing plant or industrial facility monitoring application), or may comprise injection of discrete fluid samples that have been drawn at periodic or random time intervals from a fluid source using, for example, a fraction collector. Aerodynamic design may not be required in this use case, but the detection device may comprise many of the same modular building blocks as those used in air sampling applications.

Furthermore, applications in the consumer-packaged goods industry may not require an HEPA filter, nor might they need a semi-permeable membrane for gas exchange. Rather, applications in this industry vertical may require an ancillary sample preparation system, and may require a more sophisticated data output that is non-compressed and feeds directly into a secondary software system.

### A Use Case Scenario—Public Space Security

In securing a location against threats from non-trusted agents, perimeter security alone is a weak solution. To be clear, here we are not referring to access control. Access control is a system which checks the agents that are allowed in a space. Location security assumes that access is already granted to non-trusted agents.

Currently, the intended secure/sterile space is closed off with a perimeter fence. Untrusted agents are scanned at a choke point or bottle neck. In an airport scenario this system creates an intense level of stress. Consequently, current data indicates that an adversary has an 80% chance of success in getting an explosive through these choke/check points within these airport security perimeters.

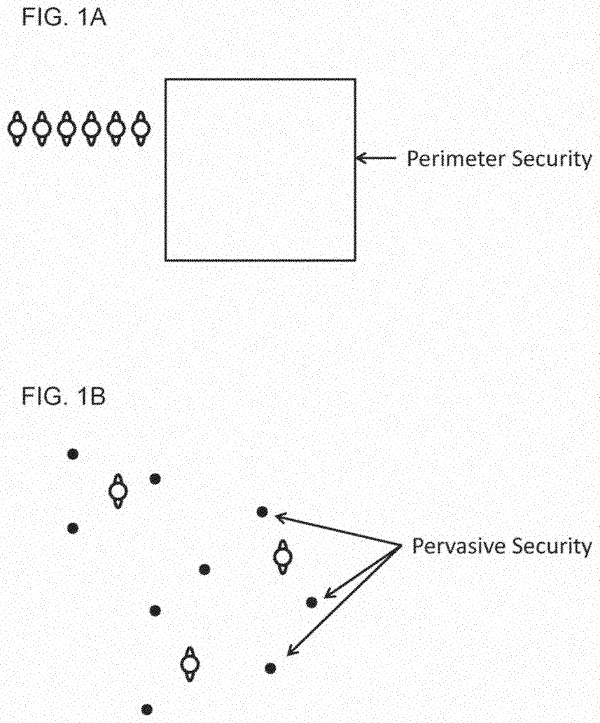

The disclosed cell-based detection devices enable a powerful paradigm shift in implementing location security. As illustrated in FIGS. 1A-B, this paradigm shift allows the space owner to choose “when, where, and how” the scan of non-trusted agents will occur. This scan is completely automated, and thus enables sophisticated backend data processing. This back end can feed into new and existing data streams to provide powerful local and global insights. The space owner can see their own local data and have access to insightful global security databases.

FIG. 1A illustrates a conventional secure perimeter approach to location security, where the focus is on restricting access. People (small symbols) line up at a checkpoint to be screened prior to gaining access to the secure space. As indicated in FIG. 1B, the disclosed devices (black circles) and systems (e.g., a network of the disclosed detection devices) return choice to the space owner or security principal. Instead of giving the choice of when to strike to an adversary, the choice of when to scan, where to scan, how to scan, and even what to scan for is returned to the system user through the use of a pervasive security system. Thus the disclosed devices and systems enable a scan on demand system.

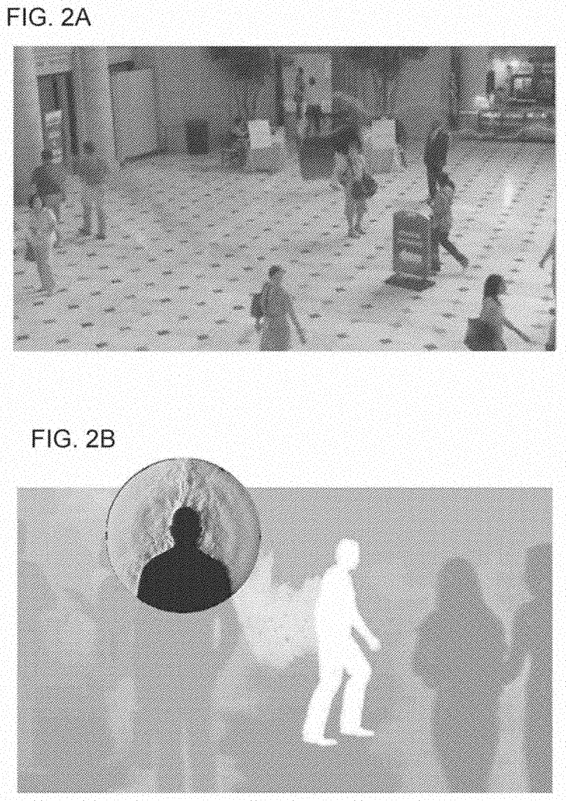

Every explosive device (excluding radiation- or high energy-based explosives), chemical or biological weapon, and contraband substance emits VOCs. The VOC particle field floats in the air surrounding the subject. In fact, the VOCs form an aura of sorts around the subject which can be viewed in a Schlieren image (a refractive gradient imaging system), as illustrated in FIG. 2A and FIG. 2B (inset). Everyone carries a sum signature of themselves and things that they are carrying, for example, food, natural odors, and importantly—explosives and contraband substances. People in a train or an airport lobby (FIG. 2A) have trails of autonomously emitted chemicals following them around. Using the disclosed devices and systems, the locations of individuals with suspect signatures can be triangulated and pin pointed. The normal heat emanating from a person encourages the development of this chemical wake (or odor wake) following behind these individuals as they move (FIG. 2B). Therefore, if an individual is carrying the source of a dangerous or illegal VOC, the trace vapor is likely included in the person's odor wake.

Naturally, the scene also contains harmless but potent odors which potentially conflate the detection of VOCs of interest. Herein resides the power of the disclosed devices and systems. These harmless VOCs constitute the background noise. During the process of receptor design or selection, airport air samples containing potentially conflating VOCs are tested to ensure that the receptors which are shipped with a detection device do not respond to background VOCs. For example, if a receptor A has a 3-fold response to caffeine and a 20-fold response to TATP (an explosive) and another receptor B has 0-fold response to caffeine but a 10-fold response to TATP, one may select the receptor B for use in the detection device.

Crowd or people flow control is another aspect which allows the system user to precisely tag the person or point of interest. The disclosed detection devices may form an important part of a much larger sensing system. Deployed with security cameras, people flow analytics, and/or biometric systems, the disclosed devices constitute a key feature of a security system. In some embodiments, the air flow through both the cell-based detection devices and/or the user environment maybe optimized to maximize detection sensitivity and allow for complete, hands-free automated solutions to public space security.

Definitions: As used herein, the singular forms “a”, “an”, and “the” include plural references unless the context clearly dictates otherwise. Any reference to “or” herein is intended to encompass “and/or” unless otherwise stated.

As used herein, the term “about” means the referenced numeric indication plus or minus 15% of that referenced numeric indication.

Samples: The term “sample” as used herein, generally refers to a sample that may or may not comprise one or more compounds. The disclosed devices and systems are generally applicable to detection of compounds (or “analytes”) in a variety of different sample types. A sample may be a gas sample (e.g., an air sample) obtained from an air space such as an outdoor air space, an air space adjacent to a factory, an air space adjacent to a deployment area of a chemical weapon, or an air space within a residential or commercial setting (i.e., from an indoor or enclosed environment).

A sample may be a liquid sample, such as a water sample obtained from a a river, a stream, a lake, an ocean, a municipal water system, or other source. A sample may be a food sample or other solid sample (which may require processing prior to the detection of compounds using the disclosed devices), or a gas or air sample drawn from a container system that houses a food sample or other solid sample.

A sample may comprise a biological sample. A biological sample may comprise urine, milk, sweat, lymph, blood, sputum, amniotic fluid, aqueous humour, vitreous humour, bile, cerebrospinal fluid, chyle, chyme, exudates, endolymph, perilymph, gastric acid, mucus, pericardial fluid, peritoneal fluid, pleural fluid, pus, rheum, saliva, sebum, serous fluid, smegma, sputum, tears, vomit, or other bodily fluid, or any combination thereof. The biological sample may comprise a fluid sample or tissue sample obtained from a subject, such as a human, animal, or plant subject. An animal subject may be, for example, a mouse, a rat, a chicken, a rabbit, a pig, a sheep, a dog, a cow, a horse, or any other animal.

A sample may be a soil sample, such as a soil sample obtained near a fracking system or oil rig. A sample may be a sample that may comprise a compound that is an environmental hazard, a health hazard, or a security hazard.

Compounds: The term “compound” as used herein, generally refers to a composition that may produce a signal in a cell, such as an electrical signal. A compound may comprise a protein, a peptide, an enzyme, an antibody, a nucleic acid, an aptamer, or a small molecule. A compound may comprise a cell or a cellular fragment. A compound may comprise a tissue or tissue fragment. A compound may comprise a naturally-derived composition or a synthetic composition.

Any of a variety of compounds may be detected using the disclosed devices as long as a suitable cell-based receptor is available, or may be designed, that exhibits a binding specificity and affinity for the compound of interest. For example, a compound may comprise an odorant molecule. A compound may comprise a compound that binds an odorant receptor or a modified odorant receptor. A compound may comprise a volatile compound. A compound may comprise an organic volatile compound. A compound may comprise a volatile molecule that provides a marker for the degree of ripeness of fruit or other agricultural products. A compound may comprise a volatile molecule that provides a marker for the degree of freshness (or spoilage) of meat or other agricultural products. A compound may comprise a neurotoxin or a toxin. A compound may comprise a cellular metabolite. A compound may comprise a carcinogen. A compound may comprise a drug or a pharmaceutical composition, or a salt thereof. A compound may comprise a marker for the health-state or disease-state of a human, animal, or plant subject. A compound may comprise an environmental pollutant. A compound may comprise a chemical weapon, such as a mustard gas, a sarin gas, or a combination thereof. A compound may comprise an illegal substance as defined in 42 United States Code § 12210. A compound may comprise an explosive, such as trinitrotoluene (TNT). A compound may be volatile marker or taggant for an explosive material. A compound may be a precursor for the compound (such as a chemical precursor), a degradation product of the compound, or a metabolite of the compound, or any combination thereof.

As noted, in some embodiments the disclosed devices and systems may be configured for the detection of one or more odorants associated with, for example, the ripeness state of fruit. Table 1 comprises a list of non-limiting examples of odorant compounds that are produced by fruit.

TABLE 1
Odorant compounds produced by fruit or plants.

| Compound Name | CAS # |
|---|---|
| alpha-ionone | 127-41-3 |
| alpha-phellandrene | 99-83-2 |
| alpha-pinene | 7785-70-8 |
| benzaldehyde | 100-52-7 |
| beta-ionone | 14901-07-6 |
| beta-pinene | 18172-67-3 |
| butyric acid | 107-92-6 |
| caryophyllen | 87-44-5 |
| damascenone | 23726-93-4 |
| delta-decalactone | 705-86-2 |
| e-2-hexenal | 6728-26-3 |
| ethyl butyrate | 105-54-4 |
| gamma-decalactone | 706-14-9 |
| geranial | 5392-40-5 |
| geraniol | 106-24-1 |
| hexanoic acid | 142-62-1 |
| hexyl acetate | 142-92-7 |
| limonene | 138-86-3 |
| linalool | 78-70-6 |
| mesifuran | 4077-47-8 |
| methyl anthranilate | 134-20-3 |
| methyl butyrate | 623-42-7 |
| neral | 5392-40-5 |
| nerolidol | 7212-44-4 |
| raspberry ketone | 5471-51-2 |

As noted, in some embodiments the disclosed devices and systems may be configured for the detection of one or more explosive compounds, or volatile markers or taggants for explosive materials. Table 2 comprises a list of non-limiting examples of volatile markers and taggants for explosive materials.

TABLE 2
Volatile markers and taggants for explosive materials.

| Compound | Description |
|---|---|
| 2,3-dimethyl-2,3-dinitrobutane | Taggant used in the U.S. for marking |
| (DMDNB/DMNB)) | plastic explosives (detectable by |
| | dogs at 0.5 ppb in air) |
| ethylene glycol dinitrate (EGDN) | Taggant used to mark Semtex |
| ortho-mononitrotoluene (o-MNT) | Taggant used for marking plastic |
| | explosives |
| para-mononitrotoluene (p-MNT) | Taggant used for marking plastic |
| | explosives |
| Dinitrotoluene (DNT) | Chemical precurson of the explosive |
| | trinitrotoluene (TNT) |
| Trinitrotoluene (TNT) | Explosive material |
| Triacetone triperoxide (TATP) | Trimer of acetone peroxide (AP) - |
| | explosive material |

Cells: The term “cell” as used herein, generally refers to one or more cells. The disclosed devices and systems may comprise one or more cells of one or more cell types. A cell may be obtained or isolated from a subject or tissue from the subject. As noted above, a subject may be a human, animal, or plant subject. A cell may be a primary cell, such as a cell or plurality of cells obtained from a brain of a subject. A cell may be a cultured cell or cultured cell line. A cell may comprise cancerous cells, non-cancerous cells, tumor cells, non-tumor cells, healthy cells, or any combination thereof In a preferred embodiment, the cells used in the disclosed devices may be neurons or other electrically-excitable cells (e.g., skeletal muscle cells, cardiac muscle cells, smooth muscle cells, and some endocrine cells, e.g., insulin-releasing pancreatic _{R} cells), as will be discussed in more detail below. In some cases, a cell may be a modified cell, such as a genetically-modified cell. A modified cell may comprise an addition and/or deletion of one of more cell-surface receptors, other cell membrane components (e.g., voltage-gated and/or ligand-gated ion channels), and/or intracellular signaling or transport components (e.g., receptor-transporting proteins). A modified cell may comprise an addition of one or more modified cell-surface receptors. The modified cell-surface receptors may be modified to increase or decrease their ability to bind to a large set of compounds, a small set of compounds, or a specific compound.

Receptors: The term “receptor” as used herein, generally refers to a receptor molecule in a cell. The receptor may be a cell-surface receptor. A cell-surface receptor may be a G-coupled protein receptor (GPCR). A receptor may bind to one or more compounds. A receptor may have a different binding affinity for each compound to which it binds. Depending on the selection of cell types and/or receptor types expressed in the cells within the device (e.g., mechanoreceptor neurons, neurons or other excitable cells expressing photoreceptors, odorant receptors, etc.), the device may be configured to sense touch, taste, sound, light, olfaction, or any combination thereof

In some instances, a receptor may be modified, such as genetically-modified. For example, a receptor may be modified to change its binding affinity for a specific compound or class of compounds. It may be modified to increase the binding affinity, or may be modified to decrease the binding affinity. In some cases, a receptor may be modified to increase its binding affinity for a specific compound or class of compounds by at least 2-fold, 3-fold, 4-fold, 5-fold, 6-fold, 7-fold, 8-fold, 9-fold, 10-fold, 15-fold, 20-fold, 25-fold, 50-fold, 75-fold, 100-fold, 500-fold, 1,000-fold or more. In some cases, a receptor may be modified to decrease its binding affinity or a specific compound or class of compounds by at least 2-fold, 3-fold, 4-fold, 5-fold, 6-fold, 7-fold, 8-fold, 9-fold, 10-fold, 15-fold, 20-fold, 25-fold, 50-fold, 75-fold, 100-fold, 500-fold, 1,000-fold or more. In some instances, a receptor may be modified to change the number of compounds to which it may bind. A receptor may be modified to increase the number of different compounds to which it may bind. A receptor may be modified to decrease the number of different compounds to which it may bind. In some cases, a receptor may bind a single compound. In some cases, a receptor may bind at least 2, 3, 4, 5, 6, 7, 8, 9, 10, 15, 20, 30, 40, 50, 60, 70, 80, 90, 100 compounds, or more. In some cases, a receptor may bind at most 100, 90, 80, 70, 60, 50, 40, 30, 20, 15, 10, 9, 8, 7, 6, 5, 4, 3, 2, or 1 compound.

The term “modification” as used herein, generally refers to a modification to a cell, a modification to a protein, or a modification to a cell receptor. A modification to a cell may include adding one or more receptors (such as modified receptors), other cell membrane components, intracellular signaling components, or intracellular transport components to the cell. A modification to a cell may include removing one or more receptors, other cell membrane components, intracellular signaling components, or intracellular transport components from a cell. A modification to a cell may include modifying one or more receptors that are expressed in the cell. A modification to a protein or cell receptor may include a genetic modification, an enzymatic modification, or a chemical modification. A modification to a protein or cell receptor may include an amino acid sequence modification (e.g., an addition, substitution, and/or deletion) or a post-translational modification such as an acylation modification, an acetylation modification, a formylation modification, an alkylation modification, a methylation modification, an arginylation modification, a polyglutamylation modification, a polyglycylation modification, a butyrylation modification, a gamma-carboxylation modification, a glycosylation modification, a malonylation modification, a hydroxylation modification, an iodination modification, a nucleotide addition modification, an oxidation modification, a phosphate ester modification, a propionylation modification, a pyroglutamate formation modification, an S-glutathionylation modification, an S-nitrosylation modification, an S-sulfenylation modification, a succinylation modification, a sulfation modification, a glycation modification, a carbamylation modification, a carbonylation modification, a biotinylation modification, a pegylation modification, or any combination thereof.

As noted, in some embodiments the disclosed devices and systems may be configured for the detection of one or more odorants associated with, for example, the ripeness state of fruit. Table 3 comprises a list of non-limiting examples of insect odorant receptors that may bind one or more of the compounds in Table 1. In some embodiments of the disclosed detection devices and systems, the cells in the devices may be engineered to express one or more of the insect odorant receptors listed in Table 3.

TABLE 3
Odorant receptors for fruit-specific volatile compounds.

| Odorant | CAS # | Organism | Literature code | GenBank ID | Literture Indication |
|---|---|---|---|---|---|
| limonene | 138-86-3 | Apolygus | AlucOR46 | NM_001190564.1 | Tuned to six plant |
| | | lucorum | | | volatiles: |
| | | (Meyer-Dür) | | | (S)-(−)- Limonene, |
| | | | | | (R)-(+)-Limonene, |
| | | | | | (E)-2- Hexenal, |
| | | | | | (E)-3-Hexenol, |
| | | | | | 1-Heptanol, and |
| | | | | | (1R)-(−)-Myrtenol |
| limonene | 138-86-3 | Megoura | OBP3 from M. | KT750882.1 | (E)-β-farnesene |
| | | viciae and | viciae | | (−)-α-pinene, |
| | | Nasonovia | | | β-pinene, and |
| | | ribisnigri | | | limonene |
| limonene | 138-86-3 | Marucavitr | MvitGOBP1-2 | NP_001140185.1 | MvitGOBP1-2 had |
| | | ata Fabricius | | | different binding |
| | | (Lepidoptera: | | | affinities with 17 volatile |
| | | Crambidae) | | | odorant molecules |
| | | | | | including butanoic acid |
| | | | | | butyl ester, limonene, 4- |
| | | | | | ethylpropiophenone, 1H |
| | | | | | indol-4-ol, butanoic acid |
| | | | | | octyl ester, and 2 methyl- |
| | | | | | 3-phenylpropanal |
| limonene | 138-86-3 | Vinegar fly | Odorant | NP_525013.2 | Single dedicated |
| | | Drosophila | receptor | | olfactory pathway |
| | | melanogaste | Or19a | | determines oviposition |
| | | r | | | fruit substrate choic |
| linalool | 78-70-6 | Bombyx | BmorOR-19 | NP_001091785.1 | Tuned to the detection of |
| | | mori | | | the plant odor linalool |

Table 4 provides a list of non-limiting examples of other odorant receptors that may be expressed in cells contained within the disclosed detection devices in order to confer compound detection specificity and sensitivity on the devices. In some cases, a cell may express multiple copies of a single odorant receptor. In some cases, each cell of a plurality of cells may express multiple copies of a single odorant receptor. In some cases, different cells (for example, cells in different fluidic chambers of the device) may express multiple copies of a different odorant receptor. A cell-based detection device may comprise cells where each odorant receptor may recognize one or more compounds, and thus the device may detect a single odorant compound or a mixture of the odorant compounds.

TABLE 4
Examples of odorant receptors

| Gene Name | Accession Number |
|---|---|
| odorant receptor family 7 subfamily D member 4 P79L variant [Homo sapiens] | ABV66285.1 |
| odorant receptor family 7 subfamily D member 4 S84N variant [Homo sapiens] | ABV66284.1 |
| odorant receptor family 7 subfamily D member 4 WM variant [Homo sapiens] | ABV66283.1 |
| odorant receptor family 7 subfamily D member 4 RT variant [Homo sapiens] | ABV66282.1 |
| odorant receptor HOR3′beta5 [Homo sapiens] | AAG42368.1 |
| odorant receptor HOR3′beta4 [Homo sapiens] | AAG42367.1 |
| odorant receptor HOR3′beta3 [Homo sapiens] | AAG42366.1 |
| odorant receptor HOR3′beta2 [Homo sapiens] | AAG42365.1 |
| odorant receptor HOR3′beta1 [Homo sapiens] | AAG42364.1 |
| olfactory receptor 7D4 [Homo sapiens] | NP_001005191.1 |
| HOR 5′Beta1 [Homo sapiens] | AAD29426.2 |
| HOR 5′Beta3 [Homo sapiens] | AAD29425.2 |
| F20722_2 [Homo sapiens] | AAC14389.1 |
| olfactory receptor 2J3 [Homo sapiens] | NP_001005216.2 |
| olfactory receptor 2H1 [Homo sapiens] | NP_001304951.1 |
| olfactory receptor 2H1 [Homo sapiens] | NP_001304943.1 |
| olfactory receptor 2H1 [Homo sapiens] | NP_112145.1 |
| olfactory receptor 11A1 [Homo sapiens] | NP_039225.1 |
| olfactory receptor 51B4 [Homo sapiens] | NP_149419.2 |
| olfactory receptor 51B2 [Homo sapiens] | NP_149420.4 |
| olfactory receptor 2J2 [Homo sapiens] | NP_112167.2 |
| olfactory receptor 2H2 [Homo sapiens] | NP_009091.3 |
| olfactory receptor 10G4 [Homo sapiens] | NP_001004462.1 |
| olfactory receptor 12D2 [Homo sapiens] | NP_039224.2 |
| olfactory receptor 2F1 [Homo sapiens] | NP_036501.2 |
| olfactory receptor 51M1 [Homo sapiens] | NP_001004756.2 |
| olfactory receptor 51I1 [Homo sapiens] | NP_001005288.1 |
| olfactory receptor 52D1 [Homo sapiens] | NP_001005163.1 |
| olfactory receptor 51I2 [Homo sapiens] | NP_001004754.1 |
| olfactory receptor 51B5 [Homo sapiens] | NP_001005567.2 |
| olfactory receptor 3A1 [Homo sapiens] | NP_002541.2 |
| olfactory receptor 51B6 [Homo sapiens] | NP_001004750.1 |
| olfactory receptor 5V1 [Homo sapiens] | NP_110503.3 |
| olfactory receptor 12D3 [Homo sapiens] | NP_112221.1 |
| olfactory receptor 10C1 [Homo sapiens] | NP_039229.3 |
| putative olfactory receptor 2B3 [Homo sapiens] | NP_001005226.1 |
| OR1F12, partial [Homo sapiens] | ADA83722.1 |
| OR12D3, partial [Homo sapiens] | ADA83721.1 |
| OR1F12, partial [Homo sapiens] | ADA83720.1 |
| F20722_1 [Homo sapiens] | AAC14388.1 |
| olfactory receptor [Homo sapiens] | CAD31042.1 |
| olfactory receptor [Homo sapiens] | CAD31041.1 |
| olfactory receptor [Homo sapiens] | CAD31040.1 |
| olfactory receptor [Homo sapiens] | CAD31039.1 |
| olfactory receptor [Homo sapiens] | CAD31038.1 |
| olfactory receptor [Homo sapiens] | CAD31037.1 |
| Olfactory receptor 51B4; Odorant receptor HOR5′beta1 | Q9Y5P0.3 |
| Olfactory receptor 51B2; Odorant receptor HOR5′beta3; Olfactory receptor 51B1 | Q9Y5P1.4 |
| Olfactory receptor 7D4; OR19-B; Odorant receptor family subfamily D member | Q8NG98.1 |
| 4RT; Olfactory receptor OR19-7 | |
| Olfactory receptor 1D2; Olfactory receptor 17-4; OR17-4; Olfactory receptor | P34982.2 |
| OR17-6; Olfactory receptor-like protein HGMP07E | |
| Olfactory receptor 12D3; Hs6M1-27; Olfactory receptor OR6-27 | Q9UGF7.1 |
| Olfactory receptor 5V1; Hs6M1-21; Olfactory receptor OR6-26 | Q9UGF6.1 |
| Olfactory receptor 11A1; Hs6M1-18; Olfactory receptor 11A2; Olfactory | Q9GZK7.1 |
| receptor OR6-30 | |
| Olfactory receptor 2H1; Hs6M1-16; OLFR42A-9004.14/9026.2; Olfactory | Q9GZK4.1 |
| receptor 2H6; Olfactory receptor 2H8; Olfactory receptor 6-2; OR6-2; Olfactory | |
| receptor OR6-32 | |
| Olfactory receptor 2J3; Hs6M1-3; Olfactory receptor OR6-16; OR6-6; Olfactory | O76001.1 |
| receptor 6-6 | |
| Receptor expression-enhancing protein 1 | Q9H902.1 |
| Receptor expression-enhancing protein 2 | Q9BRK0.2 |
| Olfactory receptor 5H8; Olfactory receptor 5H8 pseudogene; Olfactory receptor | P0DN80.1 |
| OR3-7 | |
| Olfactory receptor 13C7 | P0DN81.1 |
| Olfactory receptor 12D1; Olfactory receptor 12D1 pseudogene | P0DN82.1 |
| Putative olfactory receptor 8G3 pseudogene; Olfactory receptor OR11-297 | P0DMU2.1 |
| Putative olfactory receptor 13C6; Olfactory receptor, family 13, subfamily C, | Q8NH95.2 |
| member 6 pseudogene; Olfactory receptor, family 13, subfamily C, member 7 | |
| pseudogene; Putative olfactory receptor 13C7 | |
| Olfactory receptor 8G5; Olfactory receptor 8G6; Olfactory receptor OR11-298 | Q8NG78.2 |
| Olfactory receptor 51M1; Odorant receptor HOR5′beta7; Olfactory receptor | Q9H341.4 |
| OR11-40 | |
| Olfactory receptor 52E5 327 aa protein | Q8NH55.2 |
| Olfactory receptor 4A5; Olfactory receptor OR11-111 | Q8NH83.4 |
| Olfactory receptor 5K1; HTPCRX10; Olfactory receptor OR3-8 | Q8NHB7.2 |
| Olfactory receptor 2C1; OLFmf3; Olfactory receptor 2C2; Olfactory receptor | O95371.3 |
| OR16-1; Olfactory receptor OR16-2 | |
| Olfactory receptor 8B3; Olfactory receptor OR11-311 | Q8NGG8.3 |
| Olfactory receptor 4M2; Olfactory receptor OR15-3 | Q8NGB6.2 |
| Olfactory receptor 2H2; Hs6M1-12; Olfactory receptor 2H3; Olfactory receptor- | O95918.2 |
| like protein FAT11 | |
| Olfactory receptor 52L1; Olfactory receptor OR11-50 | Q8NGH7.4 |
| Olfactory receptor 2A14; OST182; Olfactory receptor 2A6; Olfactory receptor | Q96R47.4 |
| OR7-12 | |
| Olfactory receptor 10C1; Hs6M1-17; Olfactory receptor 10C2 | Q96KK4.3 |
| Olfactory receptor 8S1 | Q8NH09.2 |
| Olfactory receptor 8J1; Olfactory receptor OR11-183 | Q8NGP2.2 |
| Olfactory receptor 6Q1; Olfactory receptor OR11-226 317 aa protein | Q8NGQ2.2 |
| Olfactory receptor 4S2; Olfactory receptor OR11-137 | Q8NH73.2 |
| Olfactory receptor 52N4; Olfactory receptor OR11-64 | Q8N |
| Olfactory receptor 52K1; Olfactory receptor OR11-8 | Q8NGK4.2 |
| Olfactory receptor 52J3; Olfactory receptor OR11-32 | Q8NH60.2 |
| Olfactory receptor 52E2 | Q8NGJ4.2 |
| Olfactory receptor 52A1; HPFH1OR; Odorant receptor HOR3′beta4; Olfactory | Q9UKL2.2 |
| receptor OR11-319 | |
| Olfactory receptor 51V1; Odorant receptor HOR3′beta1; Olfactory receptor | Q9H2C8.2 |
| 51A12; Olfactory receptor OR11-36 | |
| Olfactory receptor 51B5; Odorant receptor HOR5′beta5; Olfactory receptor | Q9H339.2 |
| OR11-37 | |
| Olfactory receptor 10A4; HP2; Olfactory receptor-like protein JCG5 | Q9H209.2 |
| Olfactory receptor 10J1; Olfactory receptor OR1-26; Olfactory receptor-like | P30954.2 |
| protein HGMP07J | |
| Olfactory receptor 4D1; Olfactory receptor 4D3; Olfactory receptor TPCR16 | Q15615.3 |
| Olfactory receptor 12D2; Hs6M1-20; Olfactory receptor OR6-28 | P58182.2 |
| Olfactory receptor 10AC1; Olfactory receptor OR7-5 | Q8NH08.2 |
| Putative olfactory receptor 3A4; Olfactory receptor 17-24; OR17-24; Olfactory | P47883.4 |
| receptor 3A5 | |
| Olfactory receptor 56A4; Olfactory receptor OR11-49 | Q8NGH8.2 |
| Olfactory receptor 52E8; Olfactory receptor OR11-54 | Q6IFG1.3 |
| Olfactory receptor 2A25; Olfactory receptor 2A27 | A4D2G3.2 |
| Olfactory receptor 4K17; Olfactory receptor OR14-29 | Q8NGC6.3 |
| Olfactory receptor 1L1; Olfactory receptor 1L2; Olfactory receptor 9-C; OR9-C; | Q8NH94.3 |
| Olfactory receptor OR9-27 | |
| Olfactory receptor 4A15; Olfactory receptor OR11-118 | Q8NGL6.3 |
| Olfactory receptor 13D1; Olfactory receptor OR9-15 | Q8NGV5.3 |
| Olfactory receptor 8B2; Olfactory receptor OR11-309 | Q96RD0.3 |
| Olfactory receptor 2T1; Olfactory receptor 1-25; OR1-25; Olfactory receptor | O43869.3 |
| OR1-61 | |
| Olfactory receptor 6K3; Olfactory receptor OR1-18 | Q8NGY3.2 |
| Olfactory receptor 4K15; Olfactory receptor OR14-20 | Q8NH41.2 |
| Olfactory receptor 2T4; Olfactory receptor OR1-60 | Q8NH00.2 |
| Olfactory receptor 1L6; Olfactory receptor 1L7; Olfactory receptor OR9-30 | Q8NGR2.2 |
| Olfactory receptor 13A1; Olfactory receptor OR10-3 | Q8NGR1.2 |
| Olfactory receptor 56B1; Olfactory receptor OR11-65 | Q8N |
| Olfactory receptor 2AK2; Olfactory receptor 2AK1; Olfactory receptor OR1-47 | Q8NG84.2 |
| 335 aa protein | |
| Olfactory receptor 3A3; Olfactory receptor 17-201; OR17-201; Olfactory | P47888.3 |
| receptor 3A6; Olfactory receptor 3A7; Olfactory receptor 3A8; Olfactory | |
| receptor OR17-22 | |
| Olfactory receptor 3A2; Olfactory receptor 17-228; OR17-228; Olfactory | P47893.3 |
| receptor OR17-14 | |
| Olfactory receptor 10R2; Olfactory receptor OR1-8 | Q8NGX6.3 |
| Olfactory receptor 52H1; Olfactory receptor OR11-45 | Q8NGJ2.3 |
| Olfactory receptor 5T2; Olfactory receptor OR11-177 | Q8NGG2.3 |
| Olfactory receptor 6S1; Olfactory receptor OR14-37 | Q8NH40.2 |
| Olfactory receptor 6K6; Olfactory receptor OR1-21 | Q8NGW6.2 |
| Olfactory receptor 5H6; Olfactory receptor OR3-11 | Q8NGV6.2 |
| Olfactory receptor 2D3; Olfactory receptor OR11-89 | Q8NGH3.2 |
| Olfactory receptor 1S2; Olfactory receptor OR11-231 | Q8NGQ3.2 |
| Olfactory receptor 52R1; Olfactory receptor OR11-22 315 aa protein | Q8NGF1.2 |
| Olfactory receptor 51F2; Olfactory receptor OR11-23 | Q8NH61.2 |
| Olfactory receptor 10S1; Olfactory receptor OR11-279 | Q8NGN2.2 |
| Olfactory receptor 52B2; Olfactory receptor OR11-70 | Q96RD2.3 |
| Olfactory receptor 52I2; Olfactory receptor OR11-12 | Q8NH67.3 |
| Olfactory receptor 52B6; Olfactory receptor OR11-47 | Q8NGF0.3 |
| Putative olfactory receptor 52L2; Olfactory receptor OR11-74 | Q8NGH6.3 |
| Olfactory receptor 2C3; Olfactory receptor 2C4; Olfactory receptor 2C5; | Q8N628.3 |
| Olfactory receptor OR1-30 | |
| Olfactory receptor 5T3; Olfactory receptor OR11-178 | Q8NGG3.3 |
| Olfactory receptor 9K2; Olfactory receptor OR12-2 | Q8NGE7.2 |
| Olfactory receptor 7G1; Olfactory receptor 19-15; OR19-15; Olfactory receptor | Q8NGA0.2 |
| OR19-8 | |
| Olfactory receptor 4N4; Olfactory receptor OR15-1; Olfactory receptor OR15-5 | Q8N0Y3.2 |
| Olfactory receptor 2K2; HTPCRH06; Olfactory receptor OR9-17 | Q8NGT1.2 |
| Olfactory receptor 1S1; Olfactory receptor OR11-232 | Q8NH92.2 |
| Olfactory receptor 1N2; Olfactory receptor OR9-23 | Q8NGR9.2 |
| Olfactory receptor 52K2; Olfactory receptor OR11-7 | Q8NGK3.2 |
| Olfactory receptor 13C3; Olfactory receptor OR9-8 | Q8NGS6.2 |
| Olfactory receptor 4A47; Olfactory receptor OR11-113 309 aa protein | Q6IF82.2 |
| Olfactory receptor 11H1; Olfactory receptor OR22-1 | Q8NG94.3 |
| Olfactory receptor 5H2; Olfactory receptor OR3-10 | Q8NGV7.3 |
| Olfactory receptor 9G4; Olfactory receptor OR11-216 | Q8NGQ1.2 |
| Olfactory receptor 8A1; OST025; Olfactory receptor OR11-318 | Q8NGG7.2 |
| Olfactory receptor 4C13; Olfactory receptor OR11-260 | Q8NGP0.2 |
| Olfactory receptor 1A1; Olfactory receptor 17-7; OR17-7; Olfactory receptor | Q9P1Q5.2 |
| OR17-11 | |
| Olfactory receptor 5AU1; Olfactory receptor OR14-38 | Q8NGC0.2 |
| Olfactory receptor 52N5; Olfactory receptor OR11-62 | Q8NH56.2 |
| Olfactory receptor 11G2; Olfactory receptor OR14-34 | Q8NGC1.2 |
| Olfactory receptor 2D2; HB2; Olfactory receptor 11-610; OR11-610; Olfactory | Q9H210.4 |
| receptor 2D1; Olfactory receptor OR11-88 | |
| Olfactory receptor 51B6; Odorant receptor HORS′beta6 | Q9H340.2 |
| Olfactory receptor 14K1; Olfactory receptor 5AY1; Olfactory receptor OR1-39 | Q8NGZ2.2 |
| Putative olfactory receptor 9A1; HSHTPRX06 | Q8NGU1.2 |
| Olfactory receptor 14A2; Olfactory receptor 5AX1; Olfactory receptor OR1-31 | Q96R54.2 |
| Olfactory receptor 56A5 | P0C7T3.1 |
| Olfactory receptor 2T7; OST723; olfactory receptor OR1-44 | P0C7T2.1 |
| Putative olfactory receptor 2W5 320 aa protein | A6NFC9.1 |
| Olfactory receptor 52W1; Olfactory receptor OR11-71 | Q6IF63.2 |
| Olfactory receptor 11H12 | B2RN74.1 |
| Olfactory receptor 51J1; Odorant receptor HORS′beta8; Olfactory receptor 51J2 | Q9H342.2 |
| Olfactory receptor 9G9 | P0C7N8.1 |
| Olfactory receptor 8U9 | P0C7N5.1 |
| Olfactory receptor 8U8 | P0C7N1.1 |
| Olfactory receptor 11H7; Olfactory receptor OR14-32 | Q8NGC8.2 |
| Olfactory receptor 1P1; Olfactory receptor 17-208; OR17-208; Olfactory receptor | Q8NH06.2 |
| OR17-9 | |
| Olfactory receptor 1E3; Olfactory receptor 17-210; OR17-210; Olfactory | Q8WZA6.2 |
| receptor OR17-7 | |
| Olfactory receptor 8J2 | Q8NGG1.2 |
| Olfactory receptor 5G3; Olfactory receptor 5G6; Olfactory receptor OR11-213 | P0C626.1 |
| Olfactory receptor 4Q2; olfactory receptor OR14-21 | P0C623.1 |
| Olfactory receptor 4E1; Olfactory receptor OR14-43 | P0C645.1 |
| Olfactory receptor 4A8; Olfactory receptor OR11-110 | P0C604.1 |
| Olfactory receptor 5AL1; Olfactory receptor OR11-184 | P0C617.1 |
| Olfactory receptor 5AC1; Olfactory receptor OR3-2 307 aa protein | P0C628.1 |
| Olfactory receptor 52Z1 | P0C646.1 |
| Olfactory receptor 10J4 | P0C629.1 |
| Olfactory receptor 4K3; Olfactory receptor OR14-14 | Q96R72.3 |
| Olfactory receptor 2T6; OST703; Olfactory receptor 2T9 | Q8NHC8.2 |
| Olfactory receptor 1B1; Olfactory receptor 9-B; OR9-B; Olfactory receptor OR9-26 | Q8NGR6.2 |
| Olfactory receptor 10X1; Olfactory receptor OR1-14 | Q8NGY0.2 |
| Olfactory receptor 51F1 319 aa protein | A6NGY5.1 |
| Olfactory receptor 2V1 | Q8NHB1.2 |
| Olfactory receptor 4C45 | A6NMZ5.1 |
| Olfactory receptor 52A4 | A6NMU1.1 |
| Olfactory receptor 5K4 | A6NMS3.1 |
| Olfactory receptor 2AG2 | A6NM03.1 |
| Olfactory receptor 5H14 | A6NHG9.1 |
| Olfactory receptor 2T8 | A6NH00.1 |
| Olfactory receptor 6C68 | A6NDL8.2 |
| Olfactory receptor 6C6 | A6NF89.1 |
| Olfactory receptor 5K3 | A6NET4.1 |
| Olfactory receptor 5H1; HTPCRX14 | A6NKK0.1 |
| Olfactory receptor 5B21 | A6NL26.1 |
| Olfactory receptor 6C76 | A6NM76.1 |
| Olfactory receptor 6C75 | A6NL08.1 |
| Olfactory receptor 6C74 | A6NCV1.1 |
| Olfactory receptor 6C70 | A6NIJ9.1 |
| Olfactory receptor 6C65 | A6NJZ3.1 |
| Olfactory receptor 5H15 | A6NDH6.1 |
| Olfactory receptor 14I1; Olfactory receptor 5BU1 | A6ND48.1 |
| Olfactory receptor 4C46 | A6NHA9.1 |
| Olfactory receptor 2AT4; Olfactory receptor OR11-265 | A6NND4.1 |
| Olfactory receptor 4F21 | O95013.2 |
| Olfactory receptor 2M5 | A3KFT3.1 |
| Olfactory receptor 2A7; Olfactory receptor OR7-18 | Q96R45.3 |
| Olfactory receptor 3A1; Olfactory receptor 17-40; OR17-40; Olfactory receptor | P47881.2 |
| OR17-15 | |
| Olfactory receptor 2J1; Hs6M1-4; Olfactory receptor 6-5; OR6-5 | Q9GZK6.2 |
| Olfactory receptor 5K2; Olfactory receptor OR3-9 | Q8NHB8.3 |
| Olfactory receptor 4D9; Olfactory receptor OR11-253 | Q8NGE8.3 |
| Olfactory receptor 10A2; HP4; Olfactory receptor OR11-86 | Q9H208.2 |
| Olfactory receptor 7C2; Olfactory receptor 19-18; OR19-18; Olfactory receptor | O60412.4 |
| 7C3; Olfactory receptor OR19-22 | |
| Olfactory receptor 5M3; Olfactory receptor OR11-191 | Q8NGP4.2 |
| Olfactory receptor 10V1; Olfactory receptor OR11-256 | Q8N |
| Olfactory receptor 2A5; Olfactory receptor 2A26; Olfactory receptor 2A8; | Q96R48.2 |
| Olfactory receptor 7-138/7-141; OR7-138; OR7-141 | |
| Olfactory receptor 1Q1; OST226; Olfactory receptor 1Q2; Olfactory receptor | Q15612.3 |
| 1Q3; Olfactory receptor 9-A; OR9-A; Olfactory receptor OR9-25; Olfactory | |
| receptor TPCR106 | |
| Olfactory receptor 6C3; HSA8 | Q9NZP0.2 |
| Olfactory receptor 6C2; HSA3 | Q9NZP2.2 |
| Olfactory receptor 6C1; OST267 | Q96RD1.2 |
| Olfactory receptor 2T3 | Q8NH03.2 |
| Olfactory receptor 2M2; OST423 | Q96R28.2 |
| Olfactory receptor 5AC2; HSA1 | Q9NZP5.2 |
| Olfactory receptor 6B2; Olfactory receptor OR2-1 | Q6IFH4.2 |
| Olfactory receptor 2A2; Olfactory receptor 2A17; Olfactory receptor OR7-11 | Q6IF42.2 |
| Olfactory receptor 4C16; Olfactory receptor OR11-135 | Q8NGL9.2 |
| Olfactory receptor 2W3; Olfactory receptor 2W8; Olfactory receptor OR1-49 | Q7Z3T1.2 |
| Olfactory receptor 8G1; Olfactory receptor OR11-281; Olfactory receptor | Q15617.2 |
| TPCR25 | |
| Olfactory receptor 52A5; Odorant receptor HOR3′beta5; Olfactory receptor | Q9H2C5.1 |
| OR11-33 | |
| Olfactory receptor 5W2; Olfactory receptor 5W3; Olfactory receptor OR11-155 | Q8NH69.1 |
| Olfactory receptor 8U1 | Q8NH10.1 |
| Olfactory receptor 2T10; Olfactory receptor OR1-64 | Q8NGZ9.1 |
| Olfactory receptor 2AJ1 | Q8NGZ0.1 |
| Olfactory receptor 52M1; Olfactory receptor OR11-11 | Q8NGK5.1 |
| Olfactory receptor 9Q2 | Q8NGE9.1 |
| Olfactory receptor 2L3 | Q8NG85.1 |
| Olfactory receptor 10K2; Olfactory receptor OR1-4 | Q6IF99.1 |
| Olfactory receptor 2T2; Olfactory receptor OR1-43 | Q6IF00.1 |
| Olfactory receptor 2T5; Olfactory receptor OR1-62 | Q6IEZ7.1 |
| Olfactory receptor 4F3/4F16/4F29; Olfactory receptor OR1-1 | Q6IEY1.1 |
| Olfactory receptor 4C11; Olfactory receptor OR11-136 | Q6IEV9.1 |
| Olfactory receptor 5M10; Olfactory receptor OR11-207 | Q6IEU7.1 |
| Olfactory receptor 2G6 | Q5TZ20.1 |
| Olfactory receptor 10J3 | Q5JRS4.1 |
| Olfactory receptor 2B11 | Q5JQS5.1 |
| Putative olfactory receptor 2W6; Olfactory receptor OR6-3; Putative olfactory | Q8NHA6.1 |
| receptor 2W7 | |
| Olfactory receptor 10G6; Olfactory receptor OR11-280 | Q8NH81.1 |
| Putative olfactory receptor 10D3; HTPCRX09; Olfactory receptor OR11-293 | Q8NH80.1 |
| Olfactory receptor 11H2; Olfactory receptor OR14-1 | Q8NH07.1 |
| Olfactory receptor 2AP1; Olfactory receptor OR12-9 | Q8NGE2.1 |
| Olfactory receptor 4C5; Olfactory receptor OR11-99 | Q8NGB2.1 |
| Olfactory receptor 7E24; Olfactory receptor OR19-14 | Q6IFN5.1 |
| Olfactory receptor 8G2; Olfactory receptor 8G4; Olfactory receptor OR11-292; | Q6IF36.1 |
| Olfactory receptor TPCR120 | |
| Olfactory receptor 2T27; Olfactory receptor OR1-67 | Q8NH04.1 |
| Olfactory receptor 5T1; Olfactory receptor OR11-179 | Q8NG75.1 |
| Olfactory receptor 4D11 | Q8N |
| Olfactory receptor 4D10; Olfactory receptor OR11-251 | Q8N |
| Olfactory receptor 2T12; Olfactory receptor OR1-57 | Q8NG77.1 |
| Olfactory receptor 51D1; Olfactory receptor OR11-14 | Q8NGF3.1 |
| Olfactory receptor 2T33; Olfactory receptor OR1-56 | Q8NG76.1 |
| Olfactory receptor 1C1; Olfactory receptor OR1-42; Olfactory receptor TPCR27 | Q15619.4 |
| Olfactory receptor 52B4; Olfactory receptor OR11-3 | Q8NGK2.2 |
| Olfactory receptor 5R1; Olfactory receptor OR11-185 | Q8NH85.1 |
| Olfactory receptor 2V2; Olfactory receptor 2V3; Olfactory receptor OR5-3 | Q96R30.3 |
| Olfactory receptor 2M4; HTPCRX18; OST710; Olfactory receptor OR1-55; | Q96R27.2 |
| Olfactory receptor TPCR100 | |
| Olfactory receptor 2T34; Olfactory receptor OR1-63 | Q8NGX1.1 |
| Olfactory receptor 6A2; Olfactory receptor 11-55; OR11-55; Olfactory receptor | O95222.2 |
| 6A1; Olfactory receptor OR11-83; hP2 olfactory receptor | |
| Olfactory receptor 10W1; Olfactory receptor OR11-236 | Q8NGF6.1 |
| Olfactory receptor 10P1; Olfactory receptor 10P2; Olfactory receptor 10P3; | Q8NGE3.1 |
| Olfactory receptor OR12-7 | |
| Olfactory receptor 14C36; Olfactory receptor 5BF1; Olfactory receptor OR1-59 | Q8NHC7.1 |
| Olfactory receptor 10AG1; Olfactory receptor OR11-160 | Q8NH19.1 |
| Olfactory receptor 2T11; Olfactory receptor OR1-65 | Q8NH01.1 |
| Olfactory receptor 5M11 | Q96RB7.2 |
| Putative olfactory receptor 1F2; OLFmf2 | Q96R84.2 |
| Olfactory receptor 4F4; HS14a-1-A; Olfactory receptor OR19-3 | Q96R69.2 |
| Olfactory receptor 4C12; Olfactory receptor OR11-259 | Q96R67.2 |
| Olfactory receptor 5B2; OST073; Olfactory receptor OR11-240 | Q96R09.3 |
| Olfactory receptor 51E1; D-GPCR; G-protein coupled receptor 164; Olfactory | Q8TCB6.1 |
| receptor 52A3; Prostate-overexpressed G protein-coupled receptor; Prostate- | |
| specific G protein-coupled receptor 2 | |
| Putative olfactory receptor 14L1; Putative olfactory receptor 5AV1 | Q8NHC6.1 |
| Olfactory receptor 14A16; Olfactory receptor 5AT1; Olfactory receptor OR1-45 | Q8NHC5.1 |
| Olfactory receptor 10J5; Olfactory receptor OR1-28 | Q8NHC4.1 |
| Olfactory receptor 1F12; Hs6M1-35P | Q8NHA8.1 |
| Olfactory receptor 2AE1; Olfactory receptor 2AE2 | Q8NHA4.1 |
| Olfactory receptor 1L3; Olfactory receptor 9-D; OR9-D; Olfactory receptor OR9-28 | Q8NH93.1 |
| Olfactory receptor 5AK2 | Q8NH90.1 |
| Putative olfactory receptor 5AK3 | Q8NH89.1 |
| Olfactory receptor 9G1; Olfactory receptor 9G5; Olfactory receptor OR11-114 | Q8NH87.1 |
| Olfactory receptor 6X1; Olfactory receptor OR11-270 | Q8NH79.1 |
| Olfactory receptor 56B4; Olfactory receptor OR11-67 | Q8NH76.1 |
| Olfactory receptor 10A6; Olfactory receptor OR11-96 | Q8NH74.1 |
| Olfactory receptor 4C6; Olfactory receptor OR11-138 | Q8NH72.1 |
| Olfactory receptor 4A16; Olfactory receptor OR11-117 | Q8NH70.1 |
| Olfactory receptor 51A7; Olfactory receptor OR11-27 | Q8NH64.1 |
| Olfactory receptor 51H1; Olfactory receptor OR11-25 | Q8NH63.1 |
| Putative olfactory receptor 52P1 | Q8NH57.2 |
| Olfactory receptor 56A3; Olfactory receptor 56A6 | Q8NH54.2 |
| Olfactory receptor 52N1; Olfactory receptor OR11-61 | Q8NH53.1 |
| Olfactory receptor 8K3; Olfactory receptor OR11-181 | Q8NH51.1 |
| Olfactory receptor 8K5; Olfactory receptor OR11-174 | Q8NH50.1 |
| Olfactory receptor 4X1; Olfactory receptor OR11-104 | Q8NH49.1 |
| Olfactory receptor 5B3; Olfactory receptor 5B13; Olfactory receptor OR11-239 | Q8NH48.1 |
| Olfactory receptor 4L1; Olfactory receptor 4L2; Olfactory receptor OR14-28 | Q8NH43.1 |
| Olfactory receptor 4K13; Olfactory receptor OR14-27 | Q8NH42.1 |
| Olfactory receptor 4C3; Olfactory receptor OR11-98 | Q8NH37.2 |
| Olfactory receptor 4F5 | Q8NH21.1 |
| Olfactory receptor 4Q3; Olfactory receptor 4Q4; Olfactory receptor OR14-3 | Q8NH05.1 |
| Olfactory receptor 2T29 315 aa protein | Q8NH02.2 |
| Olfactory receptor 6F1; Olfactory receptor OR1-38 | Q8NGZ6.1 |
| Olfactory receptor 2G2; Olfactory receptor OR1-32 | Q8NGZ5.1 |
| Olfactory receptor 2G3; Olfactory receptor OR1-33 309 aa protein | Q8NGZ4.1 |
| Olfactory receptor 13G1; Olfactory receptor OR1-37 | Q8NGZ3.1 |
| Olfactory receptor 2L8; Olfactory receptor OR1-46 | Q8NGY9.1 |
| Putative olfactory receptor 10J6 | Q8NGY7.1 |
| Olfactory receptor 6N2; Olfactory receptor OR1-23 | Q8NGY6.1 |
| Olfactory receptor 6N1 | Q8NGY5.1 |
| Olfactory receptor 6K2; Olfactory receptor OR1-17 | Q8NGY2.1 |
| Olfactory receptor 10Z1; Olfactory receptor OR1-15 | Q8NGY1.1 |
| Olfactory receptor 6P1; Olfactory receptor OR1-12 | Q8NGX9.1 |
| Olfactory receptor 6Y1; Olfactory receptor 6Y2; Olfactory receptor OR1-11 | Q8NGX8.1 |
| Olfactory receptor 10K1; Olfactory receptor OR1-6 | Q8NGX5.1 |
| Olfactory receptor 10T2; Olfactory receptor OR1-3 | Q8NGX3.1 |
| Olfactory receptor 11L1 | Q8NGX0.1 |
| Olfactory receptor 2Y1; Olfactory receptor ORS-2 | Q8NGV0.1 |
| Putative olfactory receptor 2I1; Putative olfactory receptor 2I2; Putative olfactory | Q8NGU4.1 |
| receptor 2I3; Putative olfactory receptor 2I4 | |
| Olfactory receptor 9A4; Olfactory receptor OR7-1 | Q8NGU2.1 |
| Olfactory receptor 2A1/2A42; Olfactory receptor OR7-16; Olfactory receptor | Q8NGT9.2 |
| OR7-19 | |
| Olfactory receptor 9A2; Olfactory receptor OR7-2 | Q8NGT5.1 |
| Olfactory receptor 13J1; Olfactory receptor OR9-2 | Q8NGT2.1 |
| Olfactory receptor 13C9; Olfactory receptor OR9-13 | Q8NGT0.1 |
| Olfactory receptor 13C2; Olfactory receptor OR9-12 | Q8NGS9.1 |
| Olfactory receptor 13C5; Olfactory receptor OR9-11 | Q8NGS8.1 |
| Olfactory receptor 13C8 | Q8NGS7.1 |
| Olfactory receptor 13F1; Olfactory receptor OR9-6 | Q8NGS4.1 |
| Olfactory receptor 1J1; Olfactory receptor OR9-18 | Q8NGS3.1 |
| Olfactory receptor 1J2; HSA5; HTPCRX15; OST044; Olfactory receptor 1J3; | Q8NGS2.1 |
| Olfactory receptor 1J5; Olfactory receptor OR9-19 | |
| Olfactory receptor 1J4; HTPCRX01; Olfactory receptor OR9-21 | Q8NGS1.1 |
| Olfactory receptor 1N1; Olfactory receptor 1-26; OR1-26; Olfactory receptor | Q8NGS0.1 |
| 1N3; Olfactory receptor OR9-22 | |
| Olfactory receptor 1L8; Olfactory receptor OR9-24 | Q8NGR8.1 |
| Olfactory receptor 1L4; OST046; Olfactory receptor 1L5; Olfactory receptor 9-E; | Q8NGR5.1 |
| OR9-E; Olfactory receptor OR9-29 | Q8NGR4.1 |
| Olfactory receptor 5C1; Olfactory receptor 5C2; Olfactory receptor 9-F; OR9-F | |
| Olfactory receptor 1K1 | Q8NGR3.1 |
| Olfactory receptor 9I1; Olfactory receptor OR11-228 | Q8NGQ6.1 |
| Olfactory receptor 9Q1 | Q8NGQ5.1 |
| Olfactory receptor 10Q1; Olfactory receptor OR11-233 | Q8NGQ4.1 |
| Olfactory receptor 5M1; OST050; Olfactory receptor OR11-208 | Q8NGP8.1 |
| Olfactory receptor 5M8; Olfactory receptor OR11-194 | Q8NGP6.1 |
| Olfactory receptor 5M9; Olfactory receptor OR11-190 | Q8NGP3.1 |
| Putative olfactory receptor 4A4; Olfactory receptor OR11-107 | Q8NGN8.1 |
| Putative olfactory receptor 10D4 | Q8NGN7.1 |
| Olfactory receptor 10G7; Olfactory receptor OR11-283 | Q8NGN6.1 |
| Olfactory receptor 10G8; Olfactory receptor OR11-282 | Q8NGN5.1 |
| Olfactory receptor 10G9; Olfactory receptor 10G10 | Q8NGN4.1 |
| Olfactory receptor 10G4; Olfactory receptor OR11-278 | Q8NGN3.1 |
| Olfactory receptor 6T1; Olfactory receptor OR11-277 | Q8NGN1.1 |
| Olfactory receptor 4D5; Olfactory receptor OR11-276 | Q8NGN0.1 |
| Olfactory receptor 8D4; Olfactory receptor OR11-275 | Q8NGM9.1 |
| Olfactory receptor 6M1; Olfactory receptor OR11-271 | Q8NGM8.1 |
| Olfactory receptor 4C15; Olfactory receptor OR11-127; Olfactory receptor | Q8NGM1.1 |
| OR11-134 | |
| Olfactory receptor 4P4; Olfactory receptor 4P3 | Q8NGL7.1 |
| Olfactory receptor 5D13; Olfactory receptor OR11-142; Olfactory receptor | Q8NGL4.2 |
| OR11-148 | |
| Olfactory receptor 5D14; Olfactory receptor OR11-141; Olfactory receptor | Q8NGL3.1 |
| OR11-150 | |
| Olfactory receptor 5L1; OST262; Olfactory receptor OR11-151 | Q8NGL2.1 |
| Olfactory receptor SD 18; Olfactory receptor OR11-143; Olfactory receptor | Q8NGL1.1 |
| OR11-152 | |
| Olfactory receptor 5L2; HTPCRX16; Olfactory receptor OR11-153 | Q8NGL0.1 |
| Olfactory receptor 5D16; Olfactory receptor OR11-154 | Q8NGK9.1 |
| Olfactory receptor 52I1; Olfactory receptor OR11-13 | Q8NGK6.2 |
| Olfactory receptor 51G1; Olfactory receptor 51G3; Olfactory receptor OR11-29 | Q8NGK1.1 |
| Olfactory receptor 51G2; Olfactory receptor OR11-28 | Q8NGK0.1 |
| Olfactory receptor 51T1; Olfactory receptor OR11-26 | Q8NGJ9.1 |
| Olfactory receptor 51S1; Olfactory receptor OR11-24 | Q8NGJ8.1 |
| Olfactory receptor 51A2 | Q8NGJ7.1 |
| Olfactory receptor 51A4 | Q8NGJ6.1 |
| Olfactory receptor 51L1; Olfactory receptor OR11-31 | Q8NG15.1 |
| Olfactory receptor 52E1 | Q8NG13.1 |
| Olfactory receptor 4D6; Olfactory receptor OR11-250 | Q8NGJ1.1 |
| Olfactory receptor 5A1; OST181; Olfactory receptor OR11-249 | Q8NGJ0.1 |
| Olfactory receptor 5AN1; Olfactory receptor OR11-244 | Q8N |
| Putative olfactory receptor 56B2 | Q8N |
| Olfactory receptor 52N2; Olfactory receptor OR11-57 | Q8N |
| Olfactory receptor 52E4; Olfactory receptor OR11-55 | Q8NGH9.1 |
| Olfactory receptor 8B12; Olfactory receptor OR11-317 | Q8NGG6.1 |
| Olfactory receptor 8K1; Olfactory receptor OR11-182 | Q8NGG5.1 |
| Olfactory receptor 8H1; Olfactory receptor OR11-180 | Q8NGG4.1 |
| Olfactory receptor 8J3; Olfactory receptor OR11-173 | Q8NGG0.1 |
| Olfactory receptor 4X2; Olfactory receptor OR11-105 | Q8NGF9.1 |
| Olfactory receptor 4B1; OST208; Olfactory receptor OR11-106 | Q8NGF8.1 |
| Olfactory receptor 5B17; Olfactory receptor 5B20; Olfactory receptor OR11-237 | Q8NGF7.1 |
| Olfactory receptor 10A7; Olfactory receptor OR12-6 | Q8NGE5.1 |
| Olfactory receptor 4K14; Olfactory receptor OR14-22 | Q8NGD5.1 |
| Olfactory receptor 4K1; Olfactory receptor OR14-19 | Q8NGD4.1 |
| Olfactory receptor 4K5; Olfactory receptor OR14-16 | Q8NGD3.1 |
| Olfactory receptor 4K2; Olfactory receptor OR14-15 | Q8NGD2.1 |
| Olfactory receptor 4N2; Olfactory receptor OR14-13; Olfactory receptor OR14-8 | Q8NGD1.1 |
| Olfactory receptor 4M1; Olfactory receptor OR14-7 | Q8NGD0.1 |
| Olfactory receptor 11H4; Olfactory receptor OR14-36 | Q8NGC9.1 |
| Olfactory receptor 11H6; Olfactory receptor OR14-35 | Q8NGC7.1 |
| Olfactory receptor 6J1; Olfactory receptor 6J2 | Q8NGC5.1 |
| Olfactory receptor 10G3; Olfactory receptor OR14-40 | Q8NGC4.1 |
| Olfactory receptor 10G2 | Q8NGC3.1 |
| Olfactory receptor 4E2; Olfactory receptor OR14-42 | Q8NGC2.1 |
| Olfactory receptor 4F6; Olfactory receptor 4F12; Olfactory receptor OR15-15 | Q8NGB9.1 |
| Olfactory receptor 4F15; Olfactory receptor OR15-14 | Q8NGB8.1 |
| Olfactory receptor 4S1; Olfactory receptor OR11-100 | Q8NGB4.1 |
| Olfactory receptor 4F17; Olfactory receptor 4F11; Olfactory receptor 4F18; | Q8NGA8.1 |
| Olfactory receptor 4F19 | |
| Olfactory receptor 10H5; Olfactory receptor OR19-25; Olfactory receptor OR19-26 | Q8NGA6.1 |
| Olfactory receptor 10H4; Olfactory receptor OR19-28 | Q8NGA5.1 |
| Putative olfactory receptor 7A2; Putative olfactory receptor 7A7 | Q8NGA2.1 |
| Olfactory receptor 1M1; Olfactory receptor 19-6; OR19-6; Olfactory receptor | Q8NGA1.1 |
| OR19-5 | |
| Olfactory receptor 7G2; OST260; Olfactory receptor 19-13; OR19-13; Olfactory | Q8NG99.1 |
| receptor OR19-6 | |
| Olfactory receptor 2Z1; Olfactory receptor 2Z2; Olfactory receptor OR19-4 | Q8NG97.1 |
| Olfactory receptor 7G3; OST085; Olfactory receptor OR19-9 | Q8NG95.1 |
| Olfactory receptor 13H1; Olfactory receptor ORX-1 | Q8NG92.1 |
| Olfactory receptor 8H2; Olfactory receptor OR11-171 | Q8N162.1 |
| Olfactory receptor 6V1; Olfactory receptor OR7-3 | Q8N148.1 |
| Olfactory receptor 8H3; Olfactory receptor OR11-172 | Q8N146.1 |
| Olfactory receptor 5AS1; Olfactory receptor OR11-168 | Q8N127.1 |
| Olfactory receptor 8I2; Olfactory receptor OR11-170 | Q8N0Y5.1 |
| Putative olfactory receptor 2B8; Hs6M1-29P | P59922.1 |
| Olfactory receptor 512; Olfactory receptor OR11-266 | Q8NH18.1 |
| Olfactory receptor 2A12; Olfactory receptor OR7-10 | Q8NGT7.1 |
| Olfactory receptor 2M7; Olfactory receptor OR1-58 | Q8NG81.1 |
| Olfactory receptor 2L5; Olfactory receptor 2L11; Olfactory receptor OR1-53 | Q8NG80.1 |
| Olfactory receptor 2L13; Olfactory receptor 2L14 | Q8N349.1 |
| Olfactory receptor 51Q1 | Q8NH59.2 |
| Olfactory receptor 2L2; HTPCRH07; Olfactory receptor 2L12; Olfactory | Q8NH16.1 |
| receptor 2L4 | |
| Olfactory receptor 2T35; Olfactory receptor OR1-66 | Q8NGX2.1 |
| Olfactory receptor 6B3; Olfactory receptor OR2-2 | Q8NGW1.1 |
| Olfactory receptor 6C4; Olfactory receptor OR12-10 | Q8NGE1.1 |
| Olfactory receptor 10AD1; Olfactory receptor OR12-1 | Q8NGE0.1 |
| Olfactory receptor 2M3; Olfactory receptor 2M6; Olfactory receptor OR1-54 | Q8NG83.1 |
| Olfactory receptor 1D4; Olfactory receptor 17-30; OR17-30 | P47884.3 |
| Olfactory receptor 7D2; HTPCRH03; Olfactory receptor 19-4; OR19-4; | Q96RA2.2 |
| Olfactory receptor OR19-10 | |
| Olfactory receptor 13C4; Olfactory receptor OR9-7 | Q8NGS5.1 |
| Olfactory receptor 5AR1; Olfactory receptor OR11-209 | Q8NGP9.1 |
| Olfactory receptor 5A2; Olfactory receptor OR11-248 | Q8N |
| Olfactory receptor 5AP2 | Q8NGF4.1 |
| Olfactory receptor 4N5; Olfactory receptor OR14-33 | Q8IXE1.1 |
| Olfactory receptor 52E6; Olfactory receptor OR11-58 | Q96RD3.2 |
| Olfactory receptor 8B4; Olfactory receptor OR11-315 | Q96RC9.2 |
| Olfactory receptor 5B12; Olfactory receptor 5B16; Olfactory receptor OR11-241 | Q96R08.2 |
| Olfactory receptor 5P3; Olfactory receptor OR11-94; Olfactory receptor-like | Q8WZ94.1 |
| protein JCG1 | |
| Olfactory receptor 5P2; Olfactory receptor-like protein JCG3 | Q8WZ92.1 |
| Olfactory receptor 8D1; OST004; Olfactory receptor 8D3; Olfactory receptor | Q8WZ84.1 |
| OR11-301; Olfactory receptor-like protein JCG9 | |
| Olfactory receptor 52D1; Odorant receptor HOR5′beta14; Olfactory receptor | Q9H346.1 |
| OR11-43 | |
| Olfactory receptor 51I2; Odorant receptor HOR5′beta12; Olfactory receptor | Q9H344.1 |
| OR11-38 | |
| Olfactory receptor 51I1; Odorant receptor HOR5′beta11; Olfactory receptor | Q9H343.1 |
| OR11-39 | |
| Olfactory receptor 10H1; Olfactory receptor OR19-27 | Q9Y4A9.1 |
| Olfactory receptor 2W1; Hs6M1-15; Olfactory receptor OR6-13 | Q9Y3N9.1 |
| Olfactory receptor 14J1; Hs6M1-28; Olfactory receptor 5U1; Olfactory receptor | Q9UGF5.1 |
| OR6-25 | |
| Olfactory receptor 2S2; Olfactory receptor OR9-3 | Q9NQN1.2 |
| Olfactory receptor 10A5; HP3; Olfactory receptor 10A1; Olfactory receptor 11- | Q9H207.1 |
| 403; OR11-403; Olfactory receptor-like protein JCG6 | |
| Olfactory receptor 2AG1; HT3; Olfactory receptor 2AG3; Olfactory receptor | Q9H205.2 |
| OR11-79 | |
| Olfactory receptor 8D2; Olfactory receptor OR11-303; Olfactory receptor-like | Q9GZM6.1 |
| protein JCG2 | |
| Olfactory receptor 2B2; Hs6M1-10; Olfactory receptor 2B9; Olfactory receptor | Q9GZK3.1 |
| 6-1; OR6-1 | |
| Olfactory receptor 7A5; Olfactory receptor OR19-17; Olfactory receptor | Q15622.2 |
| TPCR92 | |
| Olfactory receptor 8B8; Olfactory receptor TPCR85; Olfactory-like receptor | Q15620.2 |
| JCG8 | |
| Olfactory receptor 10A3; HTPCRX12; Olfactory receptor OR11-97 | P58181.1 |
| Olfactory receptor 4D2; B-lymphocyte membrane protein BC2009; Olfactory | P58180.1 |
| receptor OR17-24 | |
| Olfactory receptor 2B6; Hs6M1-32; Olfactory receptor 2B1; Olfactory receptor | P58173.1 |
| 2B5; Olfactory receptor 5-40; ORS-40; Olfactory receptor 6-31; OR6-31; | |
| Olfactory receptor OR6-4 | |
| Olfactory receptor IDS; Olfactory receptor 17-31; OR17-31 | P58170.1 |
| Olfactory receptor 5F1; Olfactory receptor 11-10; OR11-10; Olfactory receptor | O95221.2 |
| OR11-167 | |
| Olfactory receptor 2A4; Olfactory receptor 2A10; Olfactory receptor OR6-37 | O95047.1 |
| Olfactory receptor 6B1; Olfactory receptor 7-3; OR7-3; Olfactory receptor OR7-9 | O95007.1 |
| Olfactory receptor 2F2; Olfactory receptor 7-1; OR7-1; Olfactory receptor OR7-6 | O95006.1 |
| Olfactory receptor 7A10; OST027; Olfactory receptor OR19-18 | O76100.1 |
| Olfactory receptor 212; Hs6M1-6; Olfactory receptor 6-8; OR6-8; Olfactory | O76002.1 |
| receptor OR6-19 | |
| Putative olfactory receptor 2B3; Hs6M1-1; Olfactory receptor OR6-14; OR6-4; | O76000.1 |
| Olfactory receptor 6-4 | |
| Olfactory receptor 1I1; Olfactory receptor 19-20; OR19-20 | O60431.1 |
| Olfactory receptor 10H3; Olfactory receptor OR19-24 | O60404.1 |
| Olfactory receptor 10H2; Olfactory receptor OR19-23 | O60403.1 |
| Olfactory receptor 7A17 | O14581.1 |
| Olfactory receptor 2F1; Olfactory receptor 2F3; Olfactory receptor 2F4; | Q13607.2 |
| Olfactory receptor 2F5; Olfactory receptor-like protein OLF3 | |
| Olfactory receptor 1G1; Olfactory receptor 17-209; OR17-209; Olfactory | P47890.2 |
| receptor 1G2; Olfactory receptor OR17-8 | |
| Olfactory receptor 1E2; Olfactory receptor 17-93/17-135/17-136; OR17-135; | P47887.2 |
| OR17-136; OR17-93; Olfactory receptor 1E4 | |
| Olfactory receptor 1A2; Olfactory receptor 17-6; OR17-6; Olfactory receptor | Q9Y585.1 |
| OR17-10 | |
| Olfactory receptor 7C1; Olfactory receptor 7C4; Olfactory receptor OR19-16; | O76099.1 |
| Olfactory receptor TPCR86 | |
| Olfactory receptor 1F1; Olfactory receptor 16-35; OR16-35; Olfactory receptor | O43749.1 |
| 1F10; Olfactory receptor 1F4; Olfactory receptor 1F5; Olfactory receptor 1F6; | |
| Olfactory receptor 1F7; Olfactory receptor 1F8; Olfactory receptor 1F9; | |
| Olfactory receptor OR16-4 | |
| Olfactory receptor 5I1; Olfactory receptor OR11-159; Olfactory receptor-like | Q13606.1 |
| protein OLF1 | |
| Olfactory receptor 1E1; Olfactory receptor 13-66; OR13-66; Olfactory receptor | P30953.1 |
| 17-2/17-32; OR17-2; OR17-32; Olfactory receptor 1E5; Olfactory receptor 1E6; | |
| Olfactory receptor 5-85; ORS-85; Olfactory receptor OR17-18; Olfactory | |
| receptor-like protein HGMP07I | |
| Olfactory receptor 56A1; Olfactory receptor OR11-75 | Q8NGH5.3 |
| putative odorant receptor 71a [Talaromyces marneffei PM1] | KFX53697.1 |
| hypothetical protein XK86_18365 [Hafnia alvei] | KKI42162.1 |
| hypothetical protein PAST3_12155 [Propionibacterium acnes HL201PA1] | KFC15621.1 |
| hypothetical protein Odosp_2381 [Odoribacter splanchnicus DSM 20712] | ADY33373.1 |
| hypothetical protein LLB_1684 [Le GIonella longbeachae D-4968] | EEZ96489.1 |
| hypothetical protein cypCar_00040615 [Cyprinus carpio] | KTG44310.1 |
| hypothetical protein cypCar_00022850 [Cyprinus carpio] | KTF94953.1 |
| hypothetical protein cypCar_00047049 [Cyprinus carpio] | KTF88600.1 |
| hypothetical protein cypCar_00047378 [Cyprinus carpio] | KTF77827.1 |
| hypothetical protein cypCar_00040594 [Cyprinus carpio] | KTF73152.1 |

Signals: The term “signal” as used herein, generally refers to a signal generated in response to a binding event, for example, a compound binding to a cell-surface receptor of a cell. The signal may be an electrical signal, such as a change in a voltage or current. The recording of a signal may comprise a voltage or a current measurement. The signal may be a change in a cell membrane potential. The signal may be a membrane depolarization. The signal may be an action potential. The signal may be an electrical signal that is subthreshold of an action potential. The signal may be a magnitude of a change in a cell membrane potential, or a magnitude of an action potential. The signal may be the number of action potentials recorded per unit time, or the occurrence of a train of action potentials. The recording of a signal may comprise measuring a signal over a period of time. Information derived from the recording of a signal may be imported into a matrix to form a fingerprint or a pattern of signals. The fingerprint or pattern of signals may be a unique fingerprint. The signal may be a measurement of a amplitude, a period, or a frequency, of a combination thereof of an electrical signal. The signal may be a time duration of a refractory period following an action potential. The signal may be a peak voltage of an action potential. The signal may be a time to a peak voltage of an action potential. The signal may be a peak voltage of a membrane depolarization. The signals generated by one or more cells, e.g., one or more neurons, in response to one or more stimuli, e.g., a ligand binding event, an electrical stimulus, or a photo-stimulus, may be recorded by the one or more electrodes of a microelectrode array which are in contact with or in close proximity to the cells of the disclosed detection devices and systems.

Applications: As noted above, the disclosed detection devices and systems may be applied to a variety of different sensing applications, and in particular, to volatile compound sensing applications. Examples include, but are not limited to, monitoring produce to determine the degree of ripeness of fruit; to detect spoilage in vegetables or other food products; to detect and diagnose disease states in patients (e.g., diabetic patients); to detect the presence of airborne toxic compounds in residential, office, or commercial spaces; or to detect volatile markers (or taggants) for explosive materials, e.g., in airport facilities. In some cases, the disclosed sensor devices and detection systems may be used for detecting a specific explosive, such as TNT and related compounds (e.g., precursor compounds, degradation products, etc.). In some cases, the disclosed sensor devices and detection systems may be used for detecting compounds that have been solubilized in any of a variety of liquid samples, for example, the detection of toxins or pollutants in water samples for environmental monitoring applications, as indicated above.

In some cases, the disclosed devices may be used to detect a compound in a direct contact mode, e.g., where the compound makes direct contact with a portion of the device. Alternatively, in some cases, the disclosed devices may be used to detect a compound through a non-contact mode, e.g., a device may be used to detect one or more degradation products or secondary metabolites rather than the primary compound, such as may occur in a hospital or residential setting.

The disclosed devices and systems may sense one or more signals or events, may control one or more signals or events, may compute an output based on one or more signals or events, or any combination thereof In some instances, the devices and systems may sense touch, taste, sound, light, olfaction, or any combination thereof.

The disclosed devices and systems may be utilized in a variety of different settings including, but not limited to, residential settings, industrial settings, public spaces (such as within an aircraft, airports, hospitals, etc.), or any combination thereof. The disclosed devices and/or systems may be deployed to provide contactless security to confirm a presence or an absence of a volatile compound in a public space. For example, they may be used for detection of compounds (such as explosive compounds) in an aircraft, an airport, or any other public space. The devices may be utilized for detection of compounds (such as mold) in a residential or commercial setting, for detection of air quality within a residential or commercial setting, or as part of a larger system that reports on air quality within a residential or commercial setting and includes a feedback mechanism for controlling one or more settings of a heating, ventilation, and air conditions (HVAC) system. The disclosed devices may be deployed as a single unit, or several devices may be deployed as part of a system and configured to communicate wirelessly or via a central wired data bus. The disclosed devices may be directly connected to another system component, or may be remotely connected to another system component. The disclosed devices may also be utilized in the food industry for detecting toxins, ripeness, food quality, flavor quality evaluation, or any combination thereof. The disclosed devices or systems may be used to provide infection tracking systems, e.g., within a hospital setting or public space, to track a spread of an infection. The disclosed devices may be utilized in an industrial setting for monitoring, for example, a manufacturing process via volatile organic compound production, monitoring air quality in a closed space, or a combination thereof. The disclosed devices may be used to detect compounds (such as volatile organic compounds) in any environment without relying on knowledge of the source of the molecule or compound of interest. The specific use case may be determined by the selection of one or more receptor characteristics that have been endowed on the cells (e.g., neurons) incorporated within the device. In some cases, the receptor characteristics of the device may be manipulated by modifying the cells and/or receptor proteins expressed therein that are incorporated within the device, e.g., through the use of genetic engineering.

Core device components: As noted above, the functional core of the disclosed cell-based detection devices comprises a microelectrode array (preferably a three-dimensional structured microelectrode array (3D-SMEA)), a microfluidics layer, and neurons that have been genetically-engineered to express one or more odorant receptors or other cell surface receptors.

The detection sensitivity of the device may be influenced by a variety of factors including, but not limited to: (i) use of a pre-concentration module to concentrate the compound of interest prior to presenting it to the neurons or other cells within the device, (ii) addition of one or more “odorant binding proteins” (e.g., soluble proteins that specific odorant molecules and improve their solubility and/or facilitate interaction with an odorant receptor) to the liquid medium bathing the cells in the device, (iii) addition of one or more compound stabilization additives (e.g., colloidal zinc) that stabilize the solubility of volatile organic compounds in solution to the liquid medium bathing the cells, (iv) genetically engineering one or more of the receptors expressed by the cells within the device to enhance binding affinity and/or the electrical response of the cell, (v) over-expressing or under-expressing the receptors in one or more of the cell types within the device, (vi) genetically engineering one or more components of the intracellular signaling pathway to tune the sensitivity and electrical response of the cells within the device, (vii) addition or genetic engineering of one or more synthetic signaling components to enhance the sensitivity and electrical response of the cells within the device, (viii) genetically deleting one or more naturally-occurring signaling components within the cells, (ix) the on-device or external electronic amplification of electrical signals recorded by the electrodes of the MEA chip, and (x) on-device or external signal processing to remove noise from the electrical signals recorded by the electrodes of the MEA chip, etc.

Neurons: In preferred embodiments, the disclosed devices and systems may comprise neurons. A neuron may be a central neuron, a peripheral neuron, a sensory neuron, an interneuron, a motor neuron, a multipolar neuron, a bipolar neuron, or a pseudo-unipolar neuron. In some embodiments, the disclosed devices and systems may comprise neuron supporting cells, such as a Schwann cells, or other types of cells. In some embodiments, the disclosed devices and systems may comprise neurons of different types such as hippocampal neurons, cortical neurons, striatal neurons, or any combination thereof In some cases, the disclosed devices and systems may comprise dopaminergic neurons.

In some embodiments, the disclosed devices and systems may comprise neurons that have been modified, e.g., genetically-modified or genetically-engineered to express a non-wild type distribution of cell surface receptors, other cell membrane components (e.g., voltage-gated and/or ligand-gated ion channels, cell surface binding components to improve cellular adhesion to the MEA, etc.), and/or intracellular signaling or transport components (e.g., receptor-transporting proteins). As noted above, a modified neuron may comprise an addition and/or deletion of one of more cell-surface receptors. A modified neuron may comprise an addition of one or more modified cell-surface receptors. The modified cell-surface receptors may be modified to increase or decrease their ability to bind to a large set of compounds, a small set of compounds, or a specific compound. The neurons of a device or system may have a tailored receptor profile. A receptor profile may be tailored to a specification application (such as infection tracking in a hospital setting or air quality detection in a residential setting) or may be tailored to one or more compounds anticipated to be detected.

In some instances, the disclosed devices may comprise a single type of neuron. In some instances, the disclosed devices may comprise two, three, four, five, six, seven, eight, nine, or ten or more different types of neurons. The neurons may be natural (e.g., wild type) cells or they may be transgenic. In some instances, the neurons may be genetically-modified cells, and may comprise foreign DNA.

In some instances, each type of neuron within the disclosed devices may express a single type of cell surface receptor. In some instances, each type of neuron within the disclosed devices may each express two, three, four, five, six, seven, eight, nine, or ten or more different types of cell surface receptors.

In some instances, the specificity of the disclosed devices may be tailored for specific applications such that, through an appropriate selection of neurons or other cell types and the number and type of cell surface receptors expressed therein, the device is able to detect the presence of a single compound of interest or a panel of compounds. In some instances, the disclosed devices may be capable of detecting and identify a single compound, two compounds, three compounds, four compounds, five compounds, six compounds, seven compounds, eight compounds, nine compounds, ten compounds, or twenty or more compounds. In some instances, the ability of the disclosed devices to detect and discriminate between compounds within a mixture of compounds in a sample may be facilitated through the use of advanced signal processing techniques using, for example, machine learning algorithms.

Microelectrode arrays (MEAs): The microelectrode arrays of the disclosed devices and systems are microfabricated chips that comprise a plurality of electrodes, and which interface with the microfluidics layer used to maintain the neurons (or other cells) within the device such that the electrodes are placed in contact with, or in close proximity to, the neurons. The plurality of electrodes in the microelectrode array (MEA) chip allows simultaneous and/or sequential recording of electrical signals produced by a plurality of neurons within the device. In some instances, the plurality of electrodes in the microelectrode array may be used to provide an electrical stimulus to the neurons within the devices, e.g., for the purpose of triggering action potentials in neurons in order to calibrate the electrical signals recorded by the measurement electrodes and/or normalize the electrical signal levels recorded for different microfluidic chambers or for microfluidic chambers comprising neurons expressing different levels and/or different types of cell surface receptors. In some embodiments, one or more electrodes in each chamber may be used to stimulate the cells to assay the health of the cells, to measure an increase in the impedance of the cell-electrode interface, or to establish a baseline reading for that particular electrode to determine what a spike train signal for stimulated cells might look like in a detection event (e.g., to establish how many cells are in close proximity or contact with the electrode, what the electrical signal waveforms from these cells look like, to prepare for bursting behavior, etc.).

In some instances, the microelectrode array may be a planar two-dimensional array of electrodes. In a preferred embodiment, the microelectrode array may comprise a three-dimensional structured microelectrode array (3D-SMEA), i.e., a microelectrode array on which the electrodes protrude above the surface plane of the substrate on which the electrodes are formed.

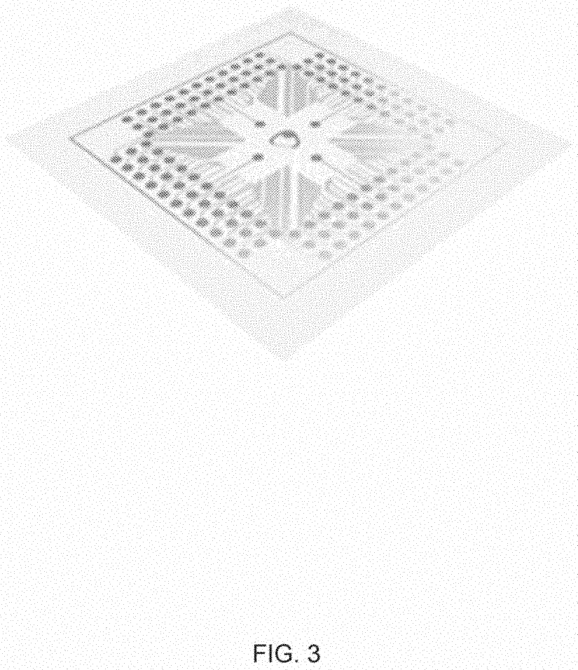

FIG. 3 illustrates a three-dimensional structured microelectrode array (3D-SMEA) that comprises a plurality of electrodes that make contact with or are positioned in close proximity to the neurons or cells within the fluid chambers of the device. In some instances, the structured microelectrode array may comprise gold electrodes patterned on a glass, silicon, or polymer substrate which is biocompatible. In other instances, the electrodes of the microelectrode array may be fabricated from any of a variety of materials known to those of skill in the art. Examples include, but are not limited to, metals, metal alloys, and metal oxides, e.g., aluminum, gold, lithium, copper, graphite, carbon, titanium, brass, silver, platinum, palladium, cesium carbonate, molybdenum(VI) oxide, indium tin oxide (ITO), or any combination thereof.

In some embodiments, the surface of the electrode may comprise a chemically-modified gold surface, wherein proteins like laminins, non-specific DNA, peptides, conductive polymers, other chemicals or compounds, or any combination thereof are grafted to the surface to improve neural adhesion and signal quality.

In some embodiments, modifying an electrode surface with a plurality of protrusions, a plurality of recesses, or by adding surface roughness may increase the surface area of the electrode and enhance contact between a cell and the electrode, thereby improving the electrical connection between the cell and the electrode.

In some embodiments, a three-dimensional electrode may comprise a spherical shape, a hemispherical shape, a mushroom shape (i.e., comprising a head portion and a support portion), a rod-like shape, a cylindrical shape, a conical shape, a patch shape, or any combination thereof.

In some embodiments, the width of an electrode (e.g., the width of the narrowest portion of a two-dimensional electrode, or the base or support portion of a three-dimensional electrode) may range from about 1 micrometer (pm) to about 50 micrometers (μm). In some embodiments, the width of an electrode may be at least 1 μm, at least 5 μm, at least 10 μm, at least 20 μm, at least 30 μm, at least 40 μm, or at least 50 μm. In some embodiments, the width of an electrode may be at most 50 μm, at most 40 μm, at most 30 μm, at most 20 μm, at most 10 μm, at most 5 μm, or at most 1 μm. Any of the lower and upper values described in this paragraph may be combined to form a range included within the present disclosure, for example, the width of an electrode may range from about 10 to about 30 Those of skill in the art will recognize that the width of an electrode may have any value within this range, e.g., about 22.5 μm.

In some embodiments, the thickness or height of an electrode (i.e., the thickness of a two-dimensional electrode, or the height of a three-dimensional electrode relative to the substrate on which it is fabricated) may range from about 0.1 micrometer (μm) to about 50 micrometers (μm). In some embodiments, the thickness or height of an electrode may be at least 0.1 at least 1 at least 5 at least 10 at least 20 at least 30 at least 40 or at least 50 In some embodiments, the thickness or height of an electrode may be at most 50 at most 40 at most 30 at most 20 at most 10 at most 5 at most 1 or at most 0.1 Any of the lower and upper values described in this paragraph may be combined to form a range included within the present disclosure, for example, the thickness or height of an electrode may range from about 0.1 to about 10 Those of skill in the art will recognize that the thickness or height of an electrode may have any value within this range, e.g., about 28.6

In some embodiments, an electrode may have a surface density of protrusions ranging from about 0.0001 protrusions per square micrometer (pro/μm²) to about 10 protrusions per square micrometer (pro/μm²). In some embodmients, the surface density of protrusions on an electrode may be at least 0.0001, at least 0.0005, at least 0.001, at least 0.002, at least 0.003, at least 0.004, at least 0.005, at least 0.006, at least 0.007, at least 0.008, at least 0.009, at least 0.01, at least 0.02, at least 0.03, at least 0.04, at least 0.05, at least 0.06, at least 0.07, at least 0.08, at least 0.09, at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 2, at least 3, at least 4, at least 5, at least 6, at least 7, at least 8, at least 9, or at least 10 protrusions per square micrometer. In some embodiments, the surface density of protrusions on an electrode may be at most 10, at most 9, at most 8, at most 7, at most 6, at most 5, at most 4, at most 3, at most 2, at most 1.5, at most 1.4, at most 1.3, at most 1.2, at most 1.1, at most 1, at most 0.9, at most 0.8, at most 0.7, at most 0.6, at most 0.5, at most 0.4, at most 0.3, at most 0.2, at most 0.1, at most 0.09, at most 0.08, at most 0.07, at most 0.06, at most 0.05, at most 0.04, at most 0.03, at most 0.02, at most 0.01, at most 0.009, at most 0.008, at most 0.007, at most 0.006, at most 0.005, at most 0.004, at most 0.003, at most 0.002, at most 0.001, at most 0.0005, or at most 0.0001 protrusions per square micrometer. Any of the lower and upper values described in this paragraph may be combined to form a range included within the present disclosure, for example, the surface density of protrusions on an electrode may range from about 0.001 to about 1.1 protrusions per square micrometer. Those of skill in the art will recognize that the surface density of protrusions on an electrode may have any value within this range, e.g., about 0.015 protrusions per square micrometer.

Similarly, in some embodiments, an electrode may have a surface density of recesses ranging from about 0.0001 recesses per square micrometer (recesses/μm²) to about 10 recesses per square micrometer (recesses/μm²). In some embodiments, the surface density of recesses on an electrode may be at least 0.0001, at least 0.0005, at least 0.001, at least 0.002, at least 0.003, at least 0.004, at least 0.005, at least 0.006, at least 0.007, at least 0.008, at least 0.009, at least 0.01, at least 0.02, at least 0.03, at least 0.04, at least 0.05, at least 0.06, at least 0.07, at least 0.08, at least 0.09, at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 2, at least 3, at least 4, at least 5, at least 6, at least 7, at least 8, at least 9, or at least 10 recesses per square micrometer. In some embodiments, the surface density of recesses on an electrode may be at most 10, at most 9, at most 8, at most 7, at most 6, at most 5, at most 4, at most 3, at most 2, at most 1.5, at most 1.4, at most 1.3, at most 1.2, at most 1.1, at most 1, at most 0.9, at most 0.8, at most 0.7, at most 0.6, at most 0.5, at most 0.4, at most 0.3, at most 0.2, at most 0.1, at most 0.09, at most 0.08, at most 0.07, at most 0.06, at most 0.05, at most 0.04, at most 0.03, at most 0.02, at most 0.01, at most 0.009, at most 0.008, at most 0.007, at most 0.006, at most 0.005, at most 0.004, at most 0.003, at most 0.002, at most 0.001, at most 0.0005, or at most 0.0001 recesses per square micrometer. Any of the lower and upper values described in this paragraph may be combined to form a range included within the present disclosure, for example, the surface density of recesses on an electrode may range from about 0.005 to about 1.6 recesses per square micrometer. Those of skill in the art will recognize that the surface density of recesses on an electrode may have any value within this range, e.g., about 0.68 recesses per square micrometer.

In some embodiments, the surface of an electrode may be smooth. In some embodiments, the surface of an electrode may have a surface roughness. A surface roughness may be uniform across the surface of an electrode. A portion of the surface of an electrode may have a surface roughness, such as a top portion of the electrode, or a bottom portion of the electrode. An electrode may have alternating rows of smooth and rough portions.

In some embodiments, a surface roughness may be about 5, 10, 15, 20, 25, 30, 35, 50, 100, 200, 300, 400, 500, 600, 700, 800, 900, 1000, 1500, 2000, 2500, 3000 nanometers (nm) or more. In some embodiments, a surface roughness may be from about 5 to about 50 nm. In some embodiments, a surface roughness may be from about 5 to about 100 nm. In some embodiments, a surface roughness may be from about 5 to about 500 nm. In some embodiments, a surface roughness may be from about 10 to about 50 nm. In some embodiments, a surface roughness may be from about 10 to about 100 nm. In some embodiments, a surface roughness may be from about 10 to about 500 nm.

In some embodiments, the MEA chip may be designed to include large numbers of electrodes distributed across a large active area. In some embodiments, the active area of the MEA chip may range from about 1 mm×1mm to about 100 mm×100 mm, or larger. In some embodiments, the active area of the MEA chip may be at least 1 mm×1 mm, at least 5 mm×5 mm, at least 10 mm×10 mm, at least 20 mm×20 mm, at least 30 mm×30 mm, at least 40 mm×40 mm, at least 50 mm×50 mm, at least 60 mm×60 mm, at least 70 mm×70 mm, at least 80 mm×80 mm, at least 90 mm×90 mm, or at least 100 mm×100 mm. In some embodiments, the active area may be square or rectangular in shape. In some embodiments, the active area may be circular or ellipsoid in shape. In some embodiments, the active area may be irregular in shape.

In some embodiments, the MEA chip may comprise between 10 and 1,000,000 electrodes. In some embodiments, the MEA chip may comprise at least 10, at least 100, at least 1,000, at least 10,000, at least 100,000, or at least 1,000,000 electrodes. In some embodiments, the electrodes of the microelectrode array may be distributed uniformly across the active area of the MEA chip, e.g., in a regular square or rectangular array. In some embodiments, the electrodes of the microelectrode array may be distributed non-uniformly across the active area of the MEA chip, e.g., clustered in areas of the active area that interface with one or more fluid chambers in the microfluidics layer.

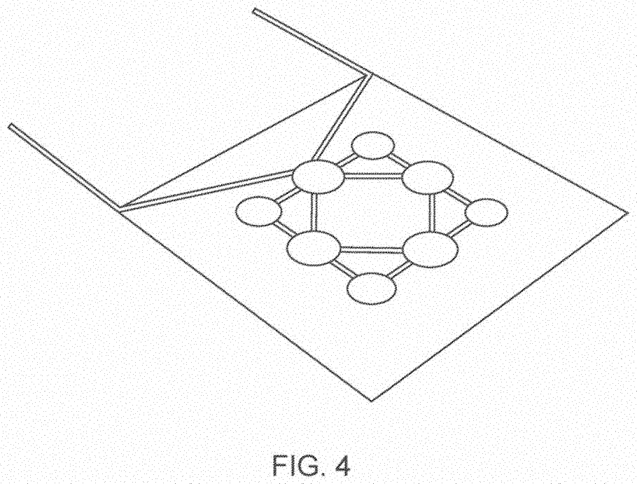

Microfluidics layer: The neurons or other cells within the device are maintained through the use of a microfluidics-based perfusion system. A system of microchannels delivers nutrients to every neuron or cell in the population of neurons or cells contained within one or more fluid chambers. As illustrated in FIG. 4, the microfluidics layer may comprise at least one fluid inlet, at least one fluid outlet, at least two microchannels, and one or more fluid chambers configured to support the growth of cells or neurons while enabling them to make contact with the underlying MEA chip. In some embodiments, the microfluidic layer may comprise at least 1, at least 2, at least 4, at least 8, at least 10, at least 20, at least 30, at least 40, at least 50, at least 60, at least 70, at least 80, at least 90, or at least 100 or more fluid chambers within which cells such as genetically-engineered neurons and/or growth medium and other fluids are contained. In some embodiments, the microfluidics layer may further comprise valves, membranes (e.g., gas exchange membranes, filter membranes, dialysis membranes, or ion exchange membranes), etc., that are fluidically coupled to one or more of the fluid chambers within the cell-based detection device. In some instances, the microfluidics system may draw and dispense components (such as fluid, gas, or a combination thereof) from or to one or more cartridges (such as replaceable, disposable cartridges) integrated into the device as will be described in more detail below. The cartridges may comprise cell culture or growth media, water, other solutions, or any combination thereof, and may be easily changeable by a user or technician.

In some embodiments, each fluid chamber of a cell-based detection device may comprise a single cell. In some embodiments, each chamber of a cell-based sensor device may comprise two cells, three cells, four cells, five cells, ten cells, twenty cells, thirty cells, forty cells, fifty cells, or more. In some embodiments, each chamber of a plurality of chambers within a cell-based sensor device may comprise the same cell or set of cells. In some embodiments, a subset of chambers or all of the chambers of a plurality of chambers with a cell-based sensor device may comprise a different cell or set of cells. The cell(s) within each chamber of the device are bathed in a cell culture medium that is continuously, periodically, or randomly perfused through each chamber of the plurality of chambers in order to maintain the viability of the cells therein. As noted above, in some instances the microfluidics layer interfaces with a semi-permeable membrane which facilitates gas exchange with an adjacent gas or air sample while containing the liquid growth medium bathing the neurons or cells within the device.

As noted above, the microfluidics layer interfaces with a microelectrode array chip comprising a plurality of electrodes. In some embodiments, there may be a single electrode in each fluid chamber of the device. In some embodiments there may be two or more electrodes in each chamber of the device. In some embodiments, there may be at least one electrode, at least two electrodes, at least three electrodes, at least four electrodes, at least five electrodes, at least six electrodes, at least seven electrodes, at least eight electrodes, at least nine electrodes, at least ten electrodes, at least twenty electrodes, at least thirty electrodes, at least forty electrodes, at least fifty electrodes, at least sixty electrodes, at least seventy electrodes, at least eighty electrodes, at least ninety electrodes, or at least one hundred electrodes in each fluid chamber of the plurality of fluid chambers within the device. In some embodiments, a single ground electrode may be placed in contact with the culture medium bathing the cells within the device. In some embodiments, at least one of the electrodes in each chamber of the plurality of chambers within the device may be a ground electrode.

The microfluidics layer of the disclosed devices may be fabricated using any of a variety of techniques and materials known to those of skill in the art. The microfluidics layer, or components thereof, may be fabricated either as monolithic parts or as an assembly of two or more separate parts that are subsequently mechanically clamped, fastened, or permanently bonded together. Examples of suitable fabrication techniques include, but are not limited to, conventional machining, CNC machining, injection molding, 3D printing, alignment and lamination of one or more layers of laser or die-cut polymer films, or any of a number of microfabrication techniques such as photolithography and wet chemical etching, dry etching, deep reactive ion etching, or laser micromachining, or any combination of these techniques. Once the microfluidics layer parts have been fabricated, they may be fastened together using any of a variety of fasteners, e.g., screws, clips, pins, brackets, and the like, or may be bonded together using any of a variety of techniques known to those of skill in the art (depending on the choice of materials used), for example, through the use of anodic bonding, thermal bonding, ultrasonic welding, or any of a variety of adhesives or adhesive films, including epoxy-based, acrylic-based, silicone-based, UV curable, polyurethane-based, or cyanoacrylate-based adhesives.

The microfluidics layer of the disclosed devices and systems may be fabricated using a variety of materials known to those of skill in the art. Examples of suitable materials include, but are not limited to, silicon, fused-silica, glass, any of a variety of polymers, e.g., polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, metals (e.g., aluminum, stainless steel, copper, nickel, chromium, and titanium), or any combination of these materials.

In some instances, fluid flow through the microfluidics-based perfusion system may be driven using an external pump, e.g., a programmable peristaltic pump or syringe pump. In some instances, fluid flow through the microfluidics-based perfusion system may be driven using a miniature pump integrated within the detection device, e.g., a microfabricated diaphragm pump or an electroosmotic pump.

Modular functional components: In addition to the core device components, in some instances the assembled devices of the present disclosure may comprise one or more additional modular functional components including, but not limited to, the following in any quantity or combination.

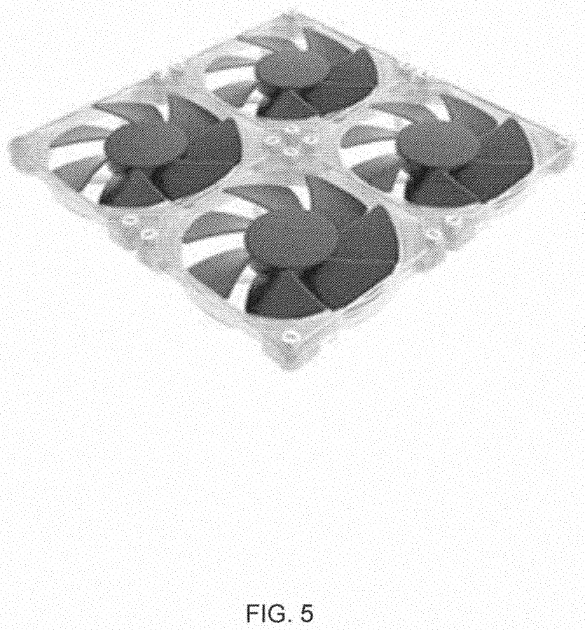

Pre-concentrator module: In some instances, the disclosed detection devices may be configured to incorporate an air pre- concentrator system. The first component of the air pre-concentrator is a specially designed fan (FIG. 5). The fan gently pulls air into the device and directs it downwards towards an air concentrator block. In combination with the design of the device housing, the fan gently pulls in air to maximize the dwell time of air containing VOCs at the semi-membrane or interface between the neurons contained within the device and the outside world. In some instances, the pre-concentrator module may further comprise at least one HEPA filter and an air director structure, as described further below.

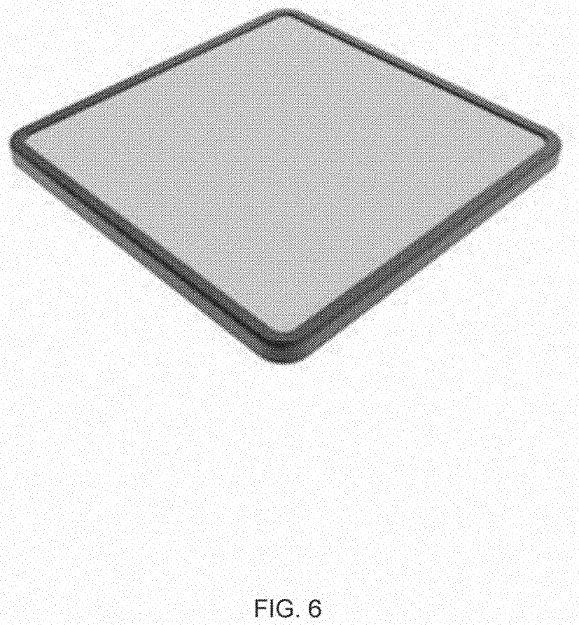

First stage HEPA filter: For many applications, the disclosed devices will be required to function in a dirty environment. In some embodiments, the devices may be configured to incorporate one or more replaceable HEPA filter that excludes dust or large, non-relevant particles from entering the next stage. FIG. 6 illustrates a serviceable HEPA filter that minimizes external environmental impact from particles and moisture. The HEPA filter used can be chosen to fit the requirements for the specific environment in which the device is to be deployed.

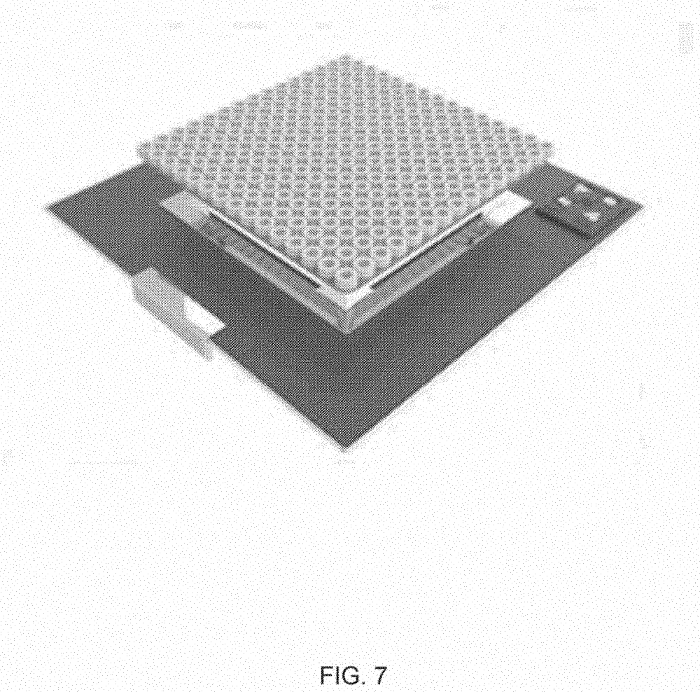

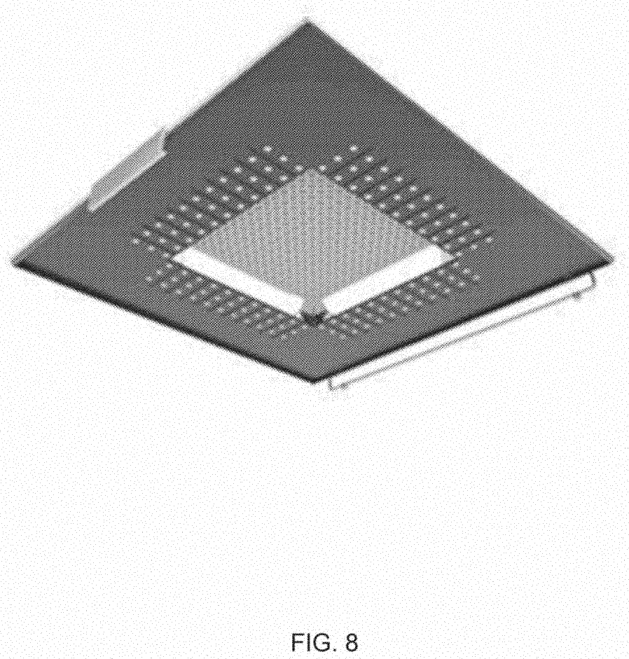

Air director: In some embodiments, the active area of the MEA chip may be relatively small. In existing prototypes, the active area is roughly 10 mm×10 mm. Therefore, the filtered, concentrated airflow drawn into the device and through the HEPA filter must be efficiently directed towards this active area for detection to occur. FIG. 7 provides a top view of an air director comprising an array of air inlets (approximately 6 cm×6 cm in area in current prototypes) that are connected via a plurality of air flow channels to an array of air outlets having a reduced area (approximately 10 mm×10 mm in current prototypes). The structure directs airflow towards the active area of the chip for detection by the cells. FIG. 8 provides a bottom view of the air director. In some instances, the air director structure may be coated with a non-stick coating to prevent or minimize adsorption of volatile compounds. The air director super structure allows for a quick exchange of the MEA chip itself within approximately 30 seconds.

In some embodiments, the air director may comprise an array of air inlets having an area ranging from about 1 cm×1 cm to about 20 cm×20 cm (or the equivalent thereof if not arranged in a regular square array). In some embodiments, the array of air inlets may have an area of at least 1 cm×1 cm, at least 2 cm×2 cm, at least 4 cm×4 cm, at least 6 cm×6 cm, at least 8 cm×8 cm, at least 10 cm×10 cm, at least 15 cm×15 cm, or at least 20 cm×20 cm or larger (or the equivalent thereof if not arranged in a regular square array). In some embodiments, the array of air inlets may comprise a regular square array or rectangular array. In some embodiments, the array of air inlets may comprise a circular array or irregular array.

In some embodiments, the air director may comprise an array of air outlets having an area ranging from about 5 mm×5 mm to about 20 mm×20 mm (or the equivalent thereof if not arranged in a regular square array). In some embodiments, the array of air outlets may have an area of at least 5 mm×5 mm, at least 7.5 mm×7.5 mm, at least 10 mm×10 mm, at least 12.5 mm×12.5 mm, at least 15 mm×15 mm, at least 17.5 mm×17.5 mm, or at least 20 mm×20 mm or larger (or the equivalent thereof if not arranged in a regular square array). In some embodiments, the array of air outlets may comprise a regular square array or rectangular array. In some embodiments, the array of air outlets may comprise a circular array or irregular array.

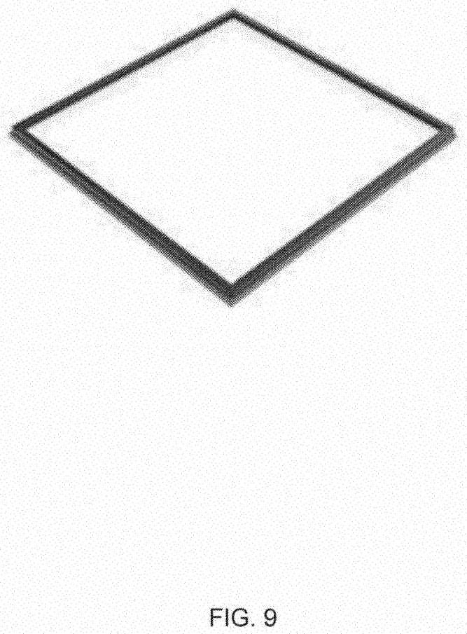

Semi permeable membrane: Compounds in fluid or gaseous samples may be introduced to the cell-based detection device either by mixing with the medium that bathes the cells in the device, or by passive diffusion (e.g., in the case of volatile compounds present in an air sample) through a semi-permeable membrane (or gas exchange memebrane) that is integrated with the device. The semi-permeable membrane (FIG. 9) is designed to promote gas exchange, thereby allowing VOCs or particles of explosives to pass through from the air sample at the outer surface to the medium bathing the cells while keeping moisture loss to a minimum. The membrane is perforated with microscopic holes, and may in some instances also be coated with a special layer to prevent explosive compounds or markers from sticking to the membrane.

In some embodiments, the semi-permeable membrane (or gas exchange membrane) may comprise a hydrophilic or hydrophobic polytetrafluoroethylene (PTFE) membrane ranging in thickness from about 10 μm to about 100 and having a pore size in the range of 0.2 to 0.5 micrometers. In some embodiments, the thickness of the hydrophobic or hydrophilic PTFE membrane may be at least 10, at least 20, at least 30, at least 40, at least 50, at least 60, at least 70, at least 80, at least 90, or at least 100 In some embodiments, the thickness of the hydrophobic or hydrophilic PTFE membrane may be at most 100, at most 90, at most 80, at most 70, at most 60, at most 50, at most 40, at most 30, at most 20, or at most 10 Any of the lower and upper values described in this paragraph may be combined to form a range included within the present disclosure, for example, the thickness of the hydrophobic or hydrophilic PTFE membrane may range from about 20 to about 80 Those of skill in the art will recognize that the thickness of the hydrophobic or hydrophilic PTFE membrane may have any value within this range, e.g., about 95 μm.

In some embodiments, the surface area-to-volume ratio for the semi-permeable membrane and the volume of liquid medium in contact with the semi-permeable membrane at a given instant is greater than 10 cm⁻¹. In some embodiments, the surface area-to-volume ratio for the semi-permeable membrane and the volume of liquid medium in contact with the semi-permeable membrane at a given instant is greater than 100 cm⁻¹. In some embodiments, the surface area-to-volume ratio for the semi-permeable membrane and the volume of liquid medium in contact with the semi-permeable membrane at a given instant is greater than 1,000 cm⁻¹.

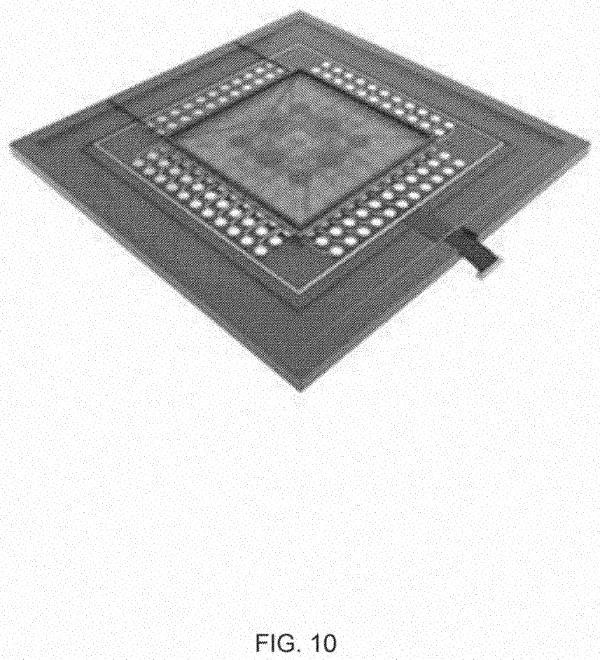

Device assembly comprising a semi permeable membrane, microfluidics layer, and three-dimensional structured MEA: The chip assembly (FIG. 10) comprising the semi-permeable membrane, microfluidics layer, and three-dimensional structured microelectrode array, constitutes the functional core of every cell-based detection device. These three individual components may be held together in fixed relative positions using a frame or fixture to form a sub-assembly of the disclosed devices.

Temperature control module: In some embodiments, the disclosed detection devices and systems may further comprise one or more temperature control components that are configured to maintain the microenvironment of the cells within the devices at a preset temperature. In some instances, the preset temperature may be any temperature within the range from about 20° C. to about 40° C. Examples of suitable temperature control module components include, but are not limited to, resistive heating elements, miniature infrared-emitting light sources, Peltier heating or cooling devices, heat sinks, thermistors, thermocouples, and the like.

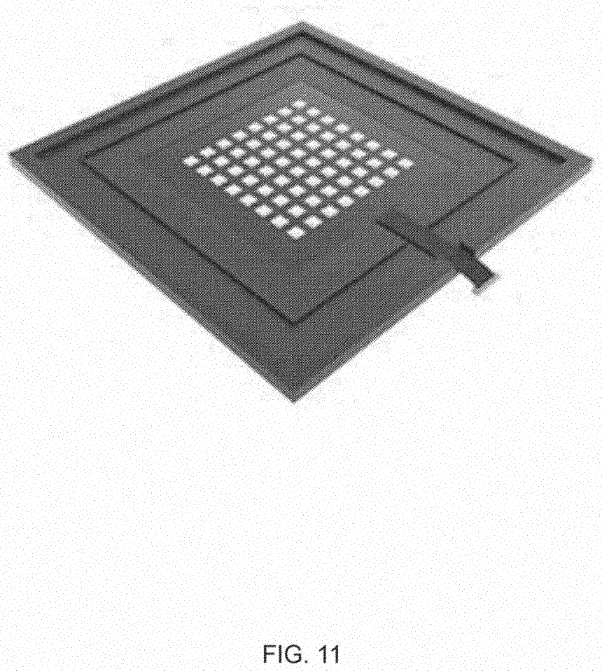

LED test layer: In many embodiments, the health of the cells may be constantly monitored to ensure optimal device performance. In some embodiments, the cells may be genetically-engineered to respond to photostimulation (e.g., by genetically incorporating light-sensitive ion channels such as a channelrhodopsin) as well as electrical stimulation. An LED array such as that depicted in FIG. 11 may be used to verify that each neuron within the device is fit for purpose, even after the device has been deployed to the field. The cells may be tested at random or at defined time intervals to check for atypical responses which, in some cases, may be corrected for during signal processing and analysis.

In general, the LED test layer will be configured so that individual LEDs or clusters of LEDs are aligned with the one or more fluid chambers within which the neurons or other cells of the device reside. The output wavelength(s) of the LEDs in the test layer should overlap with the absorption spectra of the light-sensitive ion channels used to confer photosensitivity to the cells. In some instances, different types of neurons or cells within the device may be modified to express different types of light-sensitive ion channels. In some instances, the different types of light-sensitive ion channels may exhibit different absorption spectra. In some instances the LED test layer may comprise two or more types of LEDS that emit light at two or more wavelengths (i.e., within two or more wavelength ranges).

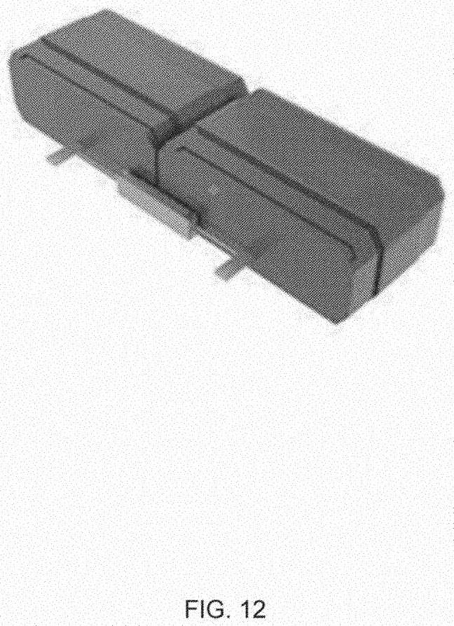

Food and waste cartridges: The neurons need to be supplied with a mix nutrients and air in order to remain viable. In some embodiments, the nutrients may be provided with any of a variety of commercially-available growth media that are known to those of skill in the art. In some embodiments, the nutrients are provided by a propriety mix. In some embodiments, the device may comprise removable cartridges for food (e.g., cell culture medium or growth medium) and/or waste (FIG. 12). The food and/or waste cartridges thus constitute a consumable component of the disclosed devices.

Typically, the cell(s) within each fluid chamber of the device are bathed in a cell culture medium that is continuously, periodically, or randomly perfused through each chamber of the plurality of chambers in order to maintain the viability of the cells therein. The medium may include one or more components, including but not limited to, sodium chloride, glycine, 1-alanine, 1-serine, a neuroactive inorganic salt, 1-aspartic acid, 1-glutamic acid, or any combination thereof. A medium may further include one or more of a pH modulating agent, an amino acid, a vitamin, a supplemental agent, a protein, an energetic substrate, a light-sensitive agent, or any combination thereof. A medium may further include one or more buffering agents. A medium may further include one or more antioxidants.

Typically, the composition and perfusion rate of the cell culture medium, as well as and other operational parameters, e.g., temperature, pH of the medium, CO₂ concentration in the medium, etc., are optimized to maintain cell viability of the cell(s) within the fluid chamber(s) of the disclosed devices. In some embodiments, the life span of the cells within the device may range from about 1 week to about 1 year. In some embodiments, the life span of cells with the device may be at least 1 week, at least 2 weeks, at least 1 month, at least 2 months, at least 3 months, at least 4 months, at least 5 months, at least 6 months, at least 7 months, at least 8 months, at least 9 months, at least 10 months, at least 11 months, or at least 1 year.

Field programmable gate arrays (FPGA), processors, and other on-board electronics: In some embodiments, the disclosed devices may be configured to incorporate an FPGA or processor chip for performing pre-processing and/or processing of electrical signals recorded by the electrodes of the MEA. In some instances, signal processing of an electrical signal measured by a single electrode, or processing of a pattern of electrical signals measured by a plurality of electrodes, enables the detection and identification of a compound or panel of compounds present in a sample.

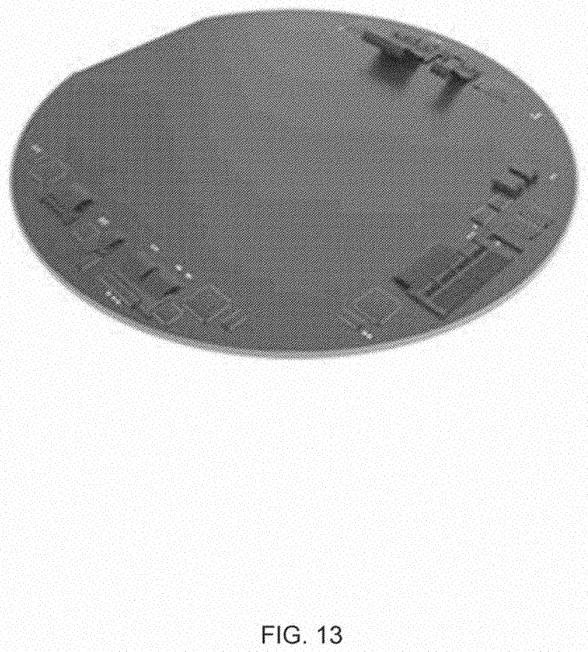

In some embodiments, all signal processing is done locally. In some embodiments, a portion of the signal processing is performed locally by the on-board FPGA or processor chip, and a portion is performed by a connected personal computer (PC), by a computer connected via a server, or by an application residing in the cloud (i.e., cloud-based computing). In some embodiments, the FPGA and/or on-board electronics also comprise a Wi-Fi module. In some embodiments, each device within a network or system of devices can be individually addressed. In some embodiments, every electrode on an MEA within the device can be individually addressed. In some embodiments, every neural subpopulation can be individually addressed including, in some cases, individual addressing of every single neuron if necessary. FIG. 13 illustrates an FPGA mounted on a frame used to assemble the modular components of the disclosed detection devices. As noted, in some embodiments, signal processing is performed locally. In some embodiments, data may be stored locally for a defined period in SRAM to avoid loss of signal, where the duration of the storage period is dependent on SRAM size. The amount of SRAM incorporated into the device may be user specific and can be customized for specific applications.

Additionally, the disclosed devices may comprise additional on-device electronics including, but not limited to, one or more signal amplifiers for amplifying one or more biological signals or events, one or more digital-to-analog converters, one or more analog-to-digital converters, a microprocessor, (such as a microprocessor programmed with software code for electrical signal spike detection), computer memory units, and the like, or any combination thereof

Machine learning-based signal processing: In some embodiments, all or a portion of the signal processing step(s) comprise the use of a machine learning algorithm for improving the accuracy of detecting and identifying compounds. Any of a variety of machine learning algorithms known to those of skill in the art may be suitable for use in processing the electrical signals generated by the cells with the disclosed detection devices and systems. Examples include, but are not limited to, supervised learning algorithms, unsupervised learning algorithms, semi-supervised learning algorithms, reinforcement learning algorithms, deep learning algorithms, or any combination thereof. In one preferred embodiment, a support vector machine learning algorithm may be used. In another preferred embodiment, a deep learning machine learning algorithm may be used.

As one non-limiting example, in some instances an artificial neural network may be used to process electrical signals recorded by the MEA. Artificial neural networks (ANN) are machine learning algorithms that may be trained to map an input data set (e.g., electrical signal patterns) to an output data set (e.g., compound identification, etc.), where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (DNN) is an ANN comprising a plurality of hidden layers, e.g., two or more hidden layers. Each layer of the neural network comprises a number of nodes (or “neurons”). A node receives input that comes either directly from the input data (e.g., sensor signals or signal patterns) or the output of nodes in previous layers, and performs a specific operation, e.g., a summation operation. In some cases, a connection from an input to a node is associated with a weight (or weighting factor). In some cases, the node may sum up the products of all pairs of inputs, xi, and their associated weights. In some cases, the weighted sum is offset with a bias, b. In some cases, the output of a node or neuron may be gated using a threshold or activation function, f, which may be a linear or non-linear function. The activation function may be, for example, a rectified linear unit (ReLU) activation function, a Leaky ReLu activation function, or other function such as a saturating hyperbolic tangent, identity, binary step, logistic, arcTan, softsign, parameteric rectified linear unit, exponential linear unit, softPlus, bent identity, softExponential, Sinusoid, Sinc, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) (e.g., a determination of compound identity and/or the position coordinates of the source of the compound) that the ANN computes are consistent with the examples included in the training data set. The parameters may be obtained from a back propagation neural network training process that may or may not be performed using the same computer system hardware as that used for performing the cell-based sensor signal processing methods disclosed herein.

Any of a variety of neural networks known to those of skill in the art may be suitable for use in processing the electrical signals generated by the cell-based detection devices and systems of the present disclosure. Examples include, but are not limited to, feedforward neural networks, radial basis function networks, recurrent neural networks, or convolutional neural networks, and the like. In some embodiments, the disclosed signal processing methods may employ a pre-trained ANN or deep learning architecture. In some embodiments, the disclosed signal processing methods may employ an ANN or deep learning architecture wherein the training data set is continuously updated with real-time detection data generated for control samples by a single local detection device, from a plurality of local detection devices (i.e., a local detection system), or from a plurality of geographically-distributed detection devices and systems that are connected through the internet.

In general, the number of nodes used in the input layer of the ANN or DNN (which may enable input of data from multiple electrodes, multiple cell-based detection devices, or multiple detection systems) may range from about 10 to about 100,000 nodes. In some instances, the number of nodes used in the input layer may be at least 10, at least 50, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, at least 20,000, at least 30,000, at least 40,000, at least 50,000, at least 60,000, at least 70,000, at least 80,000, at least 90,000, or at least 100,000. In some instances, the number of node used in the input layer may be at most 100,000, at most 90,000, at most 80,000, at most 70,000, at most 60,000, at most 50,000, at most 40,000, at most 30,000, at most 20,000, at most 10,000, at most 9000, at most 8000, at most 7000, at most 6000, at most 5000, at most 4000, at most 3000, at most 2000, at most 1000, at most 900, at most 800, at most 700, at most 600, at most 500, at most 400, at most 300, at most 200, at most 100, at most 50, or at most 10. Those of skill in the art will recognize that the number of nodes used in the input layer may have any value within this range, for example, about 512 nodes.

In some instance, the total number of layers used in the ANN or DNN (including input and output layers) may range from about 3 to about 20. In some instance the total number of layer may be at least 3, at least 4, at least 5, at least 10, at least 15, or at least 20. In some instances, the total number of layers may be at most 20, at most 15, at most 10, at most 5, at most 4, or at most 3. Those of skill in the art will recognize that the total number of layers used in the ANN may have any value within this range, for example, 8 layers.

In some instances, the total number of learnable or trainable parameters, e.g., weighting factors, biases, or threshold values, used in the ANN or DNN may range from about 1 to about 10,000. In some instances, the total number of learnable parameters may be at least 1, at least 10, at least 100, at least 500, at least 1,000, at least 2,000, at least 3,000, at least 4,000, at least 5,000, at least 6,000, at least 7,000, at least 8,000, at least 9,000, or at least 10,000. Alternatively, the total number of learnable parameters may be any number less than 100, any number between 100 and 10,000, or a number greater than 10,000. In some instances, the total number of learnable parameters may be at most 10,000, at most 9,000, at most 8,000, at most 7,000, at most 6,000, at most 5,000, at most 4,000, at most 3,000, at most 2,000, at most 1,000, at most 500, at most 100 at most 10, or at most 1. Those of skill in the art will recognize that the total number of learnable parameters used may have any value within this range, for example, about 2,200 parameters.

ANN or DNN training data sets: The input data for training of the ANN or deep learning algorithm may comprise a variety of input values and data types depending on whether the machine learning algorithm is used for processing electrical signal data for a single cell-based detection device or a system comprising a plurality of detection devices of the present disclosure. For processing electrical signals generated by individual cell-based detection devices, the input data of the training data set may comprise single time point data or multi-time point (i.e., kinetic) data for the electrical signals (e.g., voltages or currents) recorded by one or more electrodes in one or more cell-based detection devices, along with the compound identities and concentrations of control samples to which the detection devices have been exposed. For processing electrical signals generated by a detection system comprising a plurality of individual detection devices, the input data of the training data set may comprise single time point or kinetic data for the electrical signals recorded by one or more electrodes in one or more cell-based detection devices, along with the time-stamp data associated with the electrical signal data, the position coordinates for the known locations of the individual detection devices, and the compound identities, diffusion coefficients, concentrations, and position coordinates for the known locations of control samples to which the detection devices of the system have been exposed. In general, the ANN or deep learning algorithm may be trained using one or more training data sets comprising the same or different sets of input and paired output (e.g., compound identity and/or source location) data.

Distributed data processing systems and cloud-based training databases: In some embodiments, the machine learning-based methods for electrical signal processing disclosed herein may be used for processing detection device data on one or more processors, computers, or computer systems that reside at a single physical/geographical location. In some embodiments, they may be deployed as part of a distributed system of computers that comprises two or more computer systems residing at two or more physical/geographical locations. Different computer systems, or components or modules thereof, may be physically located in different workspaces and/or worksites (i.e., in different physical/geographical locations), and may be linked via a local area network (LAN), an intranet, an extranet, or the internet so that training data and/or sensor data from, e.g., air samples, to be processed may be shared and exchanged between the sites.

In some embodiments, training data may reside in a cloud-based database that is accessible from local and/or remote computer systems on which the machine learning-based sensor signal processing algorithms are running. As used herein, the term “cloud-based” refers to shared or sharable storage of electronic data. The cloud-based database and associated software may be used for archiving electronic data, sharing electronic data, and analyzing electronic data. In some embodiments, training data generated locally may be uploaded to a cloud-based database, from which it may be accessed and used to train other machine learning-based detection systems at the same site or a different site. In some embodiments, detection device and system test results generated locally may be uploaded to a cloud-based database and used to update the training data set in real time for continuous improvement of detection device and detection system test performance.

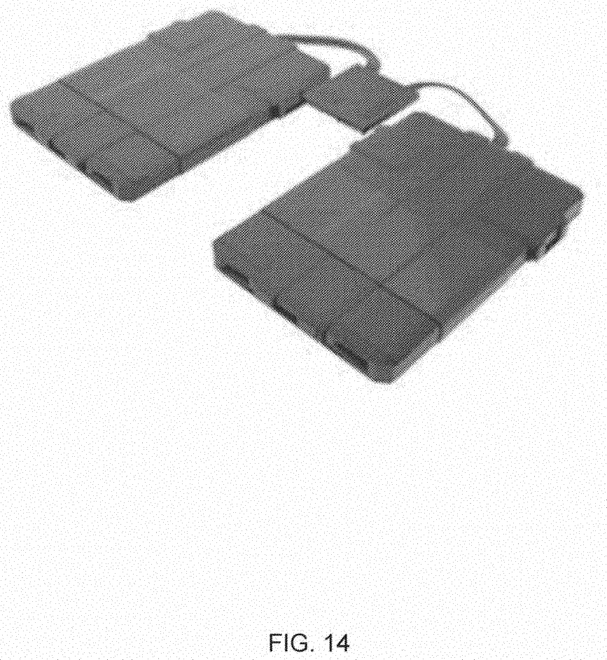

Battery: In some embodiments, a standard cell phone battery is supplied with each detection device. In some cases, a more specialized battery may be utilized that is customized to a specific use case. In some cases, a cell phone may last for up to 48 hours without requiring a charge. FIG. 14 illustrates a battery pack for use in some embodiments of the disclosed devices. In some cases, the devices can also function on a regular power supply depending on the specific use case for which they are to be deployed. The battery power required does not exceed 3.7V at 2200 mAh—which may be provided by a standard Li-ion cell phone battery.

Sensors: In some embodiments, the cell-based detection devices of the present disclosure, or one or more individual fluid chambers of a plurality of chambers contained therein, may further comprise one or more additional components for use in regulating the microenvironment of the cells within the device and maintaining cell viability. Examples include, but are not limited to, heating elements, cooling elements, temperature sensors, pH sensors, gas sensors (e.g., O₂ sensors, CO₂ sensors), glucose sensors, optical sensors, electrochemical sensors, optoelectronic sensors, piezoelectric sensors, magnetic stirring/mixing components (e.g., micro stir bars or magnetic beads that are driven by an external magnetic field), etc., or any combination thereof In some embodiments, the cell-based detection devices of the present disclosure may further comprise additional components or features, e.g., transparent optical windows, micro-lens components, or light-guiding features to facilitate microscopic observation or spectroscopic monitoring techniques, inlet and outlet ports for making connections to perfusion systems, electrical connections for connecting electrodes or sensors to external processors or power supplies, etc. In some embodiments, the disclosed sensor devices may further comprise a controller (separately or in addition to the processor discussed above) configured to control heating and/or cooling elements, and/or to send instructions to and/or read data from one or more sensors.

Frames and assembly fixtures: In some embodiments, one or more modular, functional components of the disclosed devices may be removably held in a fixed position relative to one or more adjacent functional components through the use of a frame or fixture to create sub-assemblies or assemblies of functional components. In some embodiments, two or more separate parts or components may be mechanically clamped, fastened, or permanently bonded together. Examples of suitable fabrication techniques for parts and components (including frames or fixtures used to assemble the components) include, but are not limited to, conventional machining, CNC machining, injection molding, 3D printing, alignment and lamination of one or more layers of laser or die-cut polymer films, or any of a number of microfabrication techniques such as photolithography and wet chemical etching, dry etching, deep reactive ion etching, or laser micromachining, or any combination of these techniques. Once the device components have been fabricated, they may be fastened together using any of a variety of fasteners, e.g., screws, clips, pins, brackets, and the like, or may be bonded together using any of a variety of techniques known to those of skill in the art (depending on the choice of materials used), for example, through the use of anodic bonding, thermal bonding, ultrasonic welding, or any of a variety of adhesives or adhesive films, including epoxy-based, acrylic-based, silicone-based, UV curable, polyurethane-based, or cyanoacrylate-based adhesives. In some embodiments, one or more modular, functional components of the detection device may be removable or interchangeable. For example, in some embodiments, the sub-assembly comprising the semi-permeable membrane (if included), the microfluidics layer, and 3D-SMEA may be removable and interchangeable.

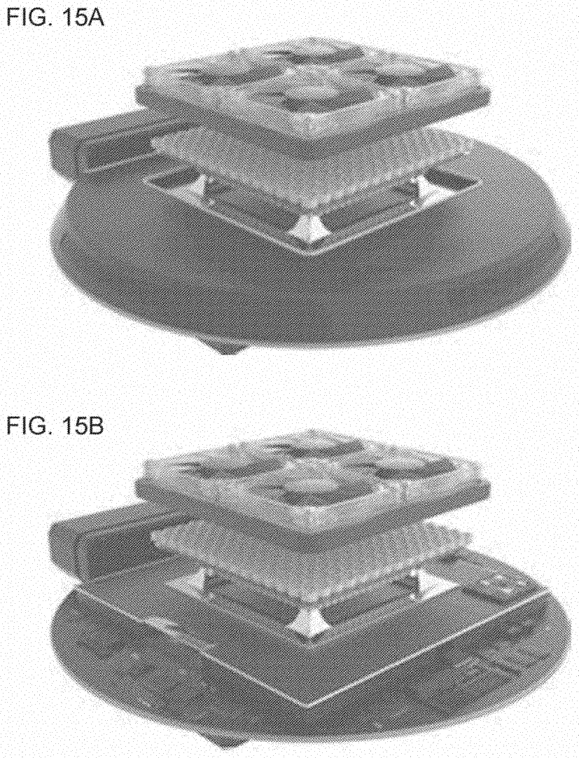

Assembled devices: FIGS. 15A-B illustrate a fully assembled device (with the device housing removed) from air intake to directing of the resultant airflow to the surface of a semi-permeable membrane. The airborne particles or VOCs cross the membrane into the medium bathing the cells, and give rise to a signal which is recorded by the electrodes of the MEA and processed by the FPGA. In some embodiments, a Wi-Fi module securely sends the processed signal and/or data to an end user, wherein the transmitted data may include the identification of the chemical detected, time stamp of detection, estimated concentration, channel, confidence level in the results, health status of the device, and other desired parameters, or any combination thereof. FIG. 15A illustrates the device with a cover plate that protects the FPGA removed. FIG. 15B illustrates the device with the cover plate installed. Prototype devices have been built and tested, and are providing guidance on how to scale up production to manufacturing levels.

Device housing: In many embodiments, the assembled detection device may comprise a housing that has one or more inlets to allow a gas (such as ambient air) to flow into the device either passively or actively, and one or more outlets to allow the gas to exit the device. In a preferred embodiment, the device housing for air-sampling applications is designed to maximize air capture and improve the detection sensitivity of the device.

In some embodiments, the device housing may comprise an outer shell component, and a baseplate (or chassis) component. In some instances, the shell component may comprise a unitary piece of material comprising a glass, polymer, ceramic, or any combination thereof. In some instances, the shell component may comprise one or more injection-molded or 3D-printed polymer parts. In some instances, the shell component may comprise an integrated HEPA filter layer for the removal of airborne particulates and contaminants that may otherwise interfere with detection of the one or more compounds of interest. In some instances, the baseplate or chassis of the device may be designed to resist vibrations or shocks that the device might be subjected (such as during use), so that it may prevent damage to the internal biologicals. In some instances, the shell component and/or baseplate of the device housing may be designed to resist or reduce electromagnetic interference and noise, which may have an impact on the process of recording electrical signals generated by the biological components of the device.

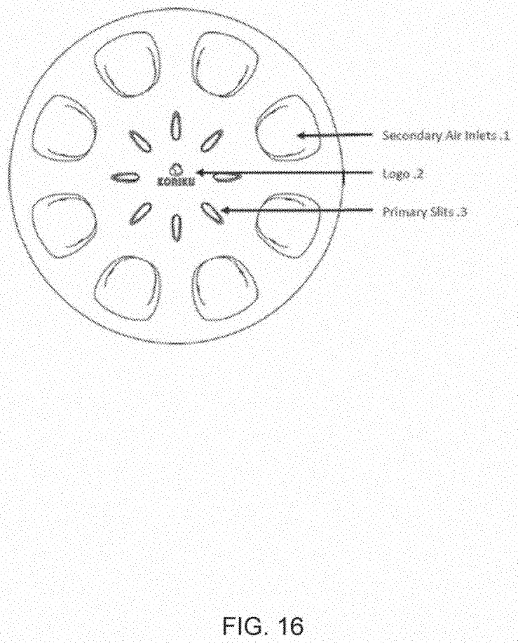

FIG. 16 shows a top view of a device housing comprising a preferred design, style, or form for the device and which contains and protects the modular, functional components of the device interior. In some instanced, the device housing may carry a logo (as illustrated in FIG. 16 at the top center of the housing). A number of radially distributed air inlet slits (e.g., 8 radially distributed slits) may allow the device to breathe or exchange air passively or actively with the external environment. Additional air inlet and/or outlet structures (such as the 8 additional structures illustrated) may be distributed radially about a center axis of the device, and may facilitate the secondary exchange of air. Active air sampling may be located at the front end of the device. In some instances, the number, shape, and position of air inlets and/or air outlets may be optimized to maximize the dwell time of the compounds to be detected within the device.

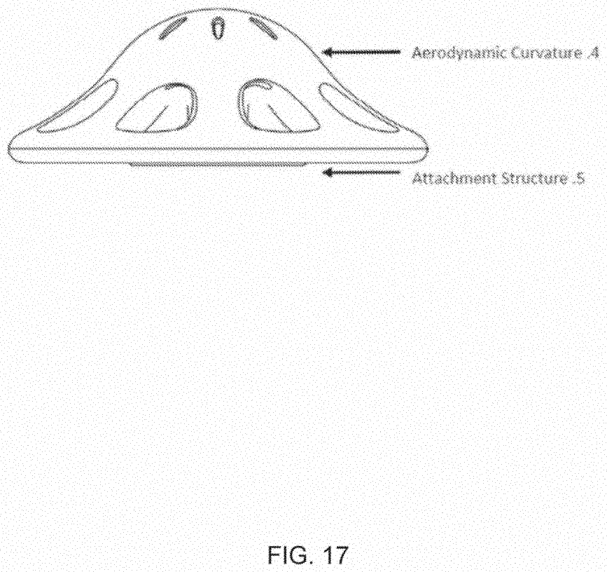

FIG. 17 shows a side view of a device housing comprising a curvature which may promote an aerodynamically efficient flow of air from the top concentric slits to the secondary air inlets. The housing may be sufficiently large to contain the neural cell culture, microfluidics layer that provides life support for the cells, a microprocessor and signal amplifier to read the information derived from the cells during a detection event, or other modular, functional components of the disclosed devices.

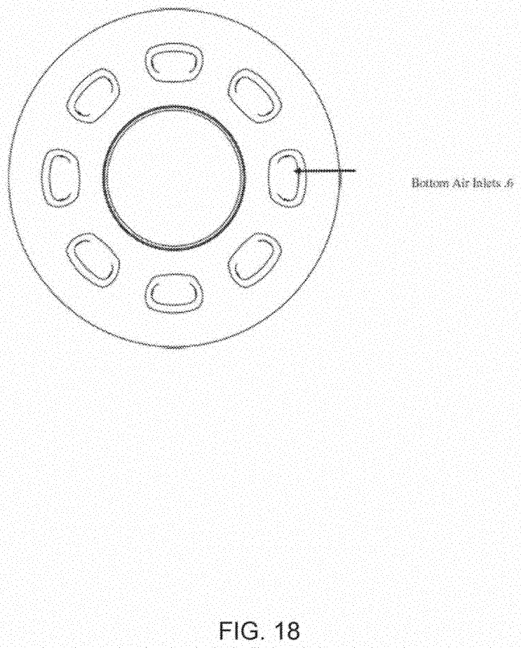

FIG. 18 shows the underside of a device housing comprising an attachment structure which may comprise an adhesive (such as Velcro), a magnetic component, a hook, a wearable attachment or any combination thereof, that may facilitate attachment of the device to a wall or other surface, a host (e.g., a human or animal), or a vehicle (e.g., a helicopter or drone).

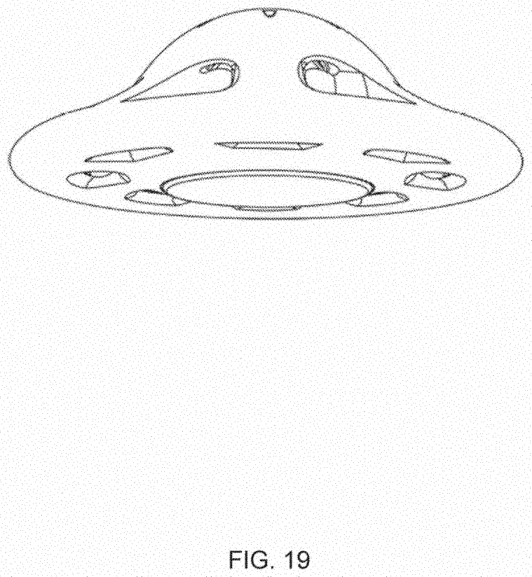

FIG. 19 shows how the underside openings of a device housing that may facilitate airflow within the device, and may facilitate a blending of secondary air flows with those entering the device through the characteristic top slits. A fan sitting in the top of the device may blow air through a filter (such as a HEPA filter) to remove ambient macroscale particulates and may blow the air directly onto a membrane (such as a PTFE membrane) covering the cell culture. The shape and positioning of the air inlets may be configured to increase beneficial air currents, increase compound dwell time at the membrane surface to improve the permeation of the compounds through the membrane and into the media, or a combination thereof. The membrane may be engineered to be permeable to the compounds of interest (such as volatile organics) while preventing rapid evaporation of the cellular media that the neurons live in to avoid an undesirable shift in osmolality. The membrane may be configured to remove smaller biological hazards such as bacteria and viruses that may not be removed by the first stage filter.

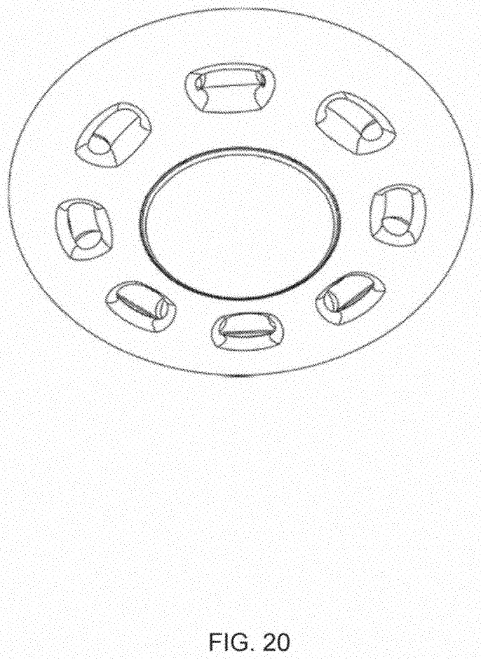

FIG. 20 shows the underside of the device housing, including the baseplate (circular component) which may comprise an attachment structure. One or more electronic ports may connect to a central wired bus, and may be positioned to allow two or more devices to be used in tandem to detect a panel of molecules, to determine the range to a source of the detected molecules, or to be used for device redundancy.

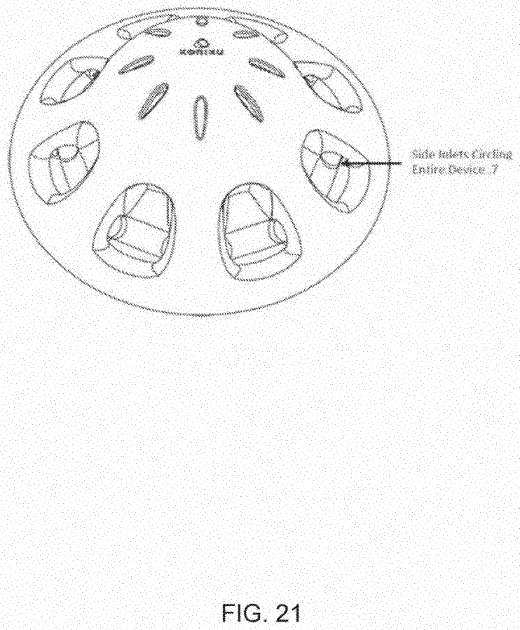

FIG. 21 illustrates a device housing comprising side inlets, 7, for air or gas samples that may circle all or a portion of the device. Some or all of the side inlets may lead to a filter membrane (such as a HEPA filter) to prevent accumulation of dust particles within the device.

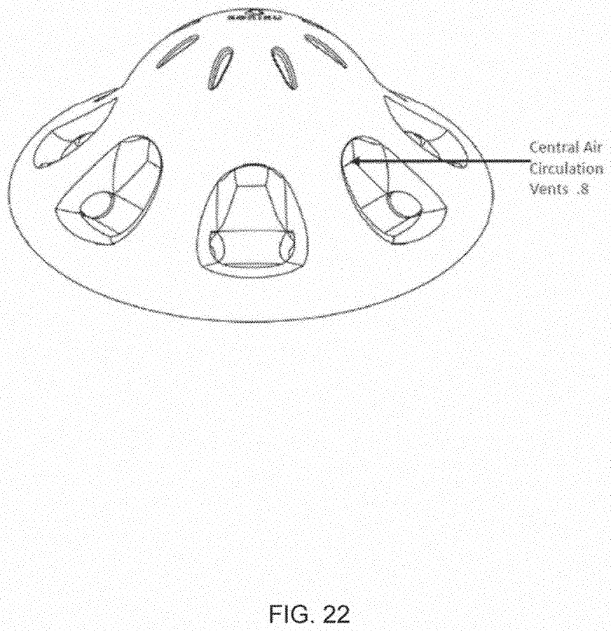

FIG. 22 illustrates a device housing comprising central air circulation vents, 8, which may be distributed symmetrically about a central axis of the device. The central air circulation vents lead to the central chamber of the device which comprises a plurality of modular functional components. This arrangement of the air inlets may allow for compounds arriving from any direction to be collected and reported, and may be specially designed to optimize the probability of the compounds of interest solvating into the detection media. In some embodiments, one or more central air circulation vents may be distributed non-symmetrically about a central axis of the device.

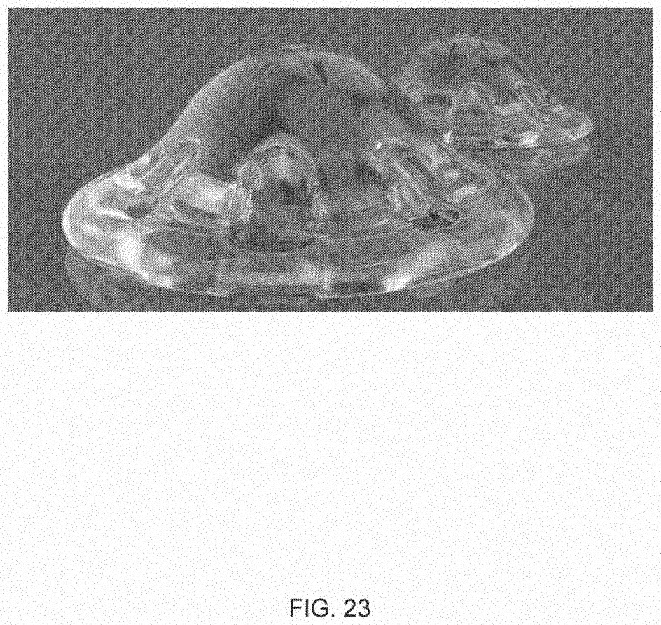

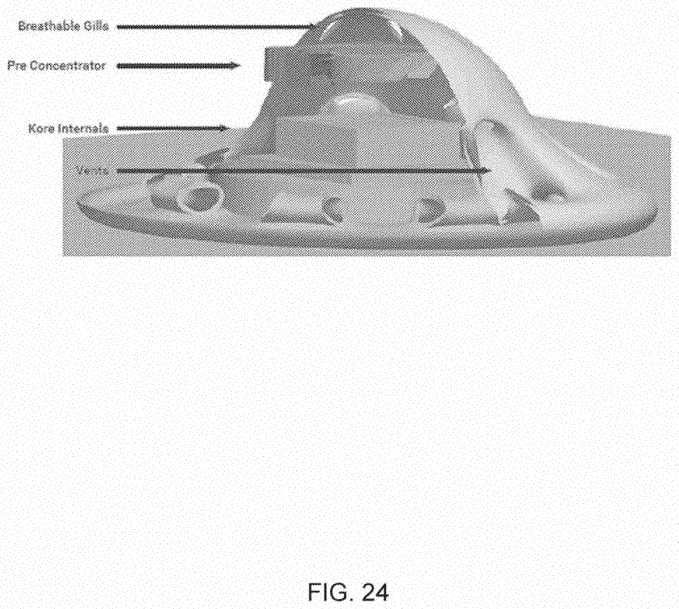

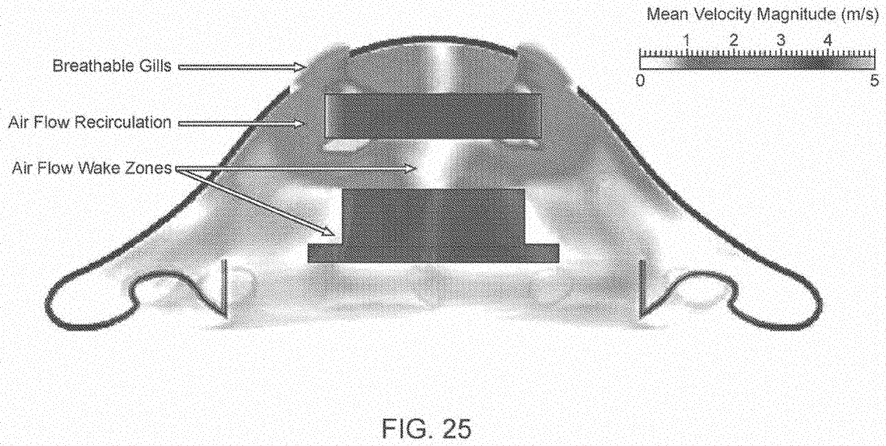

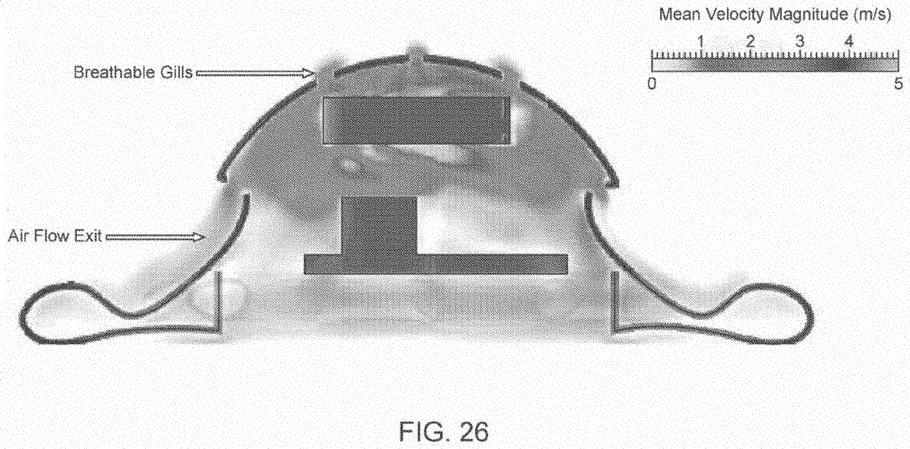

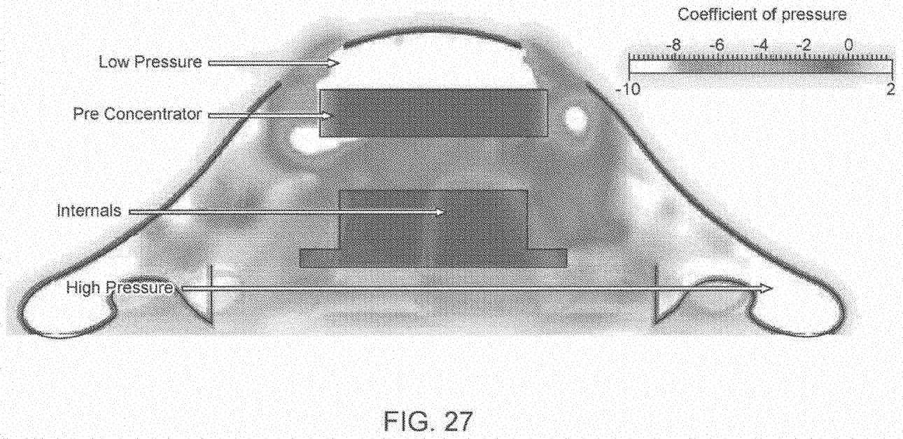

FIG. 23 illustrates a design in which a HEPA filter (textured surface) is bonded to the surface of the housing to exclude large particles from interfering with the normal function of the device. The top side is covered with gills (i.e., air inlet slits) which allow the device to draw in air to be samples, and the side of the housing comprises vents (i.e., air outlets) with a dual purpose of venting air and ensuring a long dwell time for particles at or near the surface of the semi-permeable membrane protecting the neurons within the microfluidic layer. FIG. 24 provides a cut-away view of the interior of an assembled device showing the placement of the modular device components in one embodiment. The device can house as many pre-concentrators as required to detect particles or VOCs in the air. FIG. 25 provides an example of simulation data for the computed air velocities and wake zones within an air-sampling compound detection device. The computed wake zone data verifies the aerodynamic and practical implication of the design. Recirculating air within the device gives small quantities of compounds several ‘passes’ or chances of detection by being recycled in the system. FIG. 26 provides an example of simulation data for the computed air velocities near the air outlets of the device that indicate laminar flow of the exiting air. The number and shape of air inlets (or gills) and air outlets (or vents) are optimized for maximum performance. FIG. 27 provides an example of computational fluid dynamics simulations that also confirm that the enhanced air pressure zone (green) directly above the semi-permeable membrane that results from the design of the device housing may facilitate compound diffusion through the membrane interface to allow permeation of the medium bathing the cells and thus enhance detection sensitivity.

In some embodiments, the device housing comprises: a) a shell component, wherein the shell component comprises: i) a structure comprising a sigmoidal shape that is rotationally symmetric about a single axis; ii) two or more air inlets positioned concentrically around the single axis; iii) two or more air outlets positioned concentrically around the single axis; and b) a baseplate component; wherein the sigmoidal shape of the shell component and positions of the two or more air inlets and two or more air outlets are configured to prolong a dwell time of molecules or particles transported into an interior of the device housing by a flow of air. In some embodiments, the baseplate component further comprises an attachment structure. In some embodiments, the attachment structure comprises a permanent adhesive (e.g., a UV curable epoxy), a non-permanent adhesive (e.g., a double-sided tape, a temporary bonding adhesive, etc.), a Velcro component, a magnetic component, a hook, a wearable attachment, or any combination thereof In some embodiments, the shell component is an injection-molded or three-dimensional printed part. In some embodiments, the shell component is fabricated from a polymer, a glass, a metal, a ceramic, or any combination thereof.

In some embodiments, the disclosed devices may comprise an outer radius that ranges from about 100 mm to about 300 mm. In some embodiments, the device may comprise an outer radius that is at least 100 mm, at least 120 mm, at least 140 mm, at least 160 mm, at least 180 mm, at least 200 mm, at least 220 mm, at least 240 mm, at least 260 mm, at least 280 mm, or at least 300 mm. In some embodiments, the device may comprise an outer radius of at most 300mm, at most 280 mm, at most 260 mm, at most 240 mm, at most 220 mm, at most 200 mm, at most 180 mm, at most 160 mm, at most 140 mm, at most 120 mm, or at most 100 mm.

The device may weigh less than about 1 kilogram. The device may weigh from about 1 gram to about 1 kilogram. The device may weigh from about 1 gram to about 0.75 kilogram. The device may weigh from about 1 gram to about 0.5 kilogram. The device may weigh from about 1 gram to about 0.25 kilogram. The device may weigh from about 1 gram to about 0.1 kilogram. The device may weigh from about 1 gram to about 0.075 kilogram. The device may weigh from about 1 gram to about 0.05 kilogram. The device may weigh from about 1 gram to about 0.025 kilogram.

Self-contained, transportable detection devices: In some configurations, the disclosed cell-based detection devices may comprise food and waste cartridges, batteries, and Wi-Fi connectivity constitute fully self-contained, transportable detection devices that may be utilized in a variety of ways, either individually or as part of a network or system, for a variety of industrial applications. In some embodiments, for example, location security, the baseplate component of the housing may comprise an attachment structure configured to attach the device to an internal or external wall, an internal or external floor, a ceiling of a room, or a roof of a building. In some embodiments, for example, surveillance or monitoring of outdoor spaces, the baseplate component of the housing may comprise an attachment structure configured to attach the device to a bicycle, motorcycle, automobile, plane, helicopter, robot, drone, or other manned or unmanned aerial vehicle. In some embodiments, for example, investigative activities, the baseplate component of the device housing may comprise an attachment structure configured to permit the device to be worn by an animal or a human.

As noted above, in some embodiments, one or more detection devices may be configured as part of a larger system which may further comprise other types of air sampling devices, liquid sampling devices, sensors, processors or computers, user interface devices, computer memory units, intranet or internet connectivity devices, WiFi connectivity devices, etc.

### Example—Airport Detection of Volatile Compounds

In one non-limiting example of an application for the disclosed devices and systems, a device may be positioned on a wall or other surface within an airport. The device may be attached to the wall or surface using, for example, an adhesive. The device may comprise a plurality of neurons genetically modified to express receptors for the detection of three different volatile organic compounds. In some instances, ambient air from the space surrounding the device may passively circulate through the device. In some instances, ambient air from the space surrounding the device may be actively drawn into or through the device, e.g., using one of the fan modules described above. When the ambient air contains, for example, at least about a 0.1 parts per million (ppm) concentration of at least one of the three different volatile organic compounds, a binding event may occur between the compound and one or more receptors within the plurality of neurons. The binding event may generate an electrical signal within one or more of the neurons that is recorded by one or more electrodes of a plurality of electrodes within an MEA chip that has been incorporated into the detection device. The electrical signal (or pattern of electrical signals) may be processed by a processor or controller of the device, and a positive indication of the presence and concentration of the volatile compound may be visually and/or audibly reported by the device to an airport security professional. In some embodiments, a plurality of said detection devices may be integrated as part of a distributed detection system which may further comprise additional functional components as described above.

Prototype devices as described above have been assembled and tested for the detection of a variety of volatile markers for explosive compounds using genetically-engineered neurons that express odorant receptors. The prototype testing data collected to-date demonstrate the ability of these cell-based devices to detect the presence of explosive compound markers in air samples with performance metrics that match or exceed those of conventional technologies.

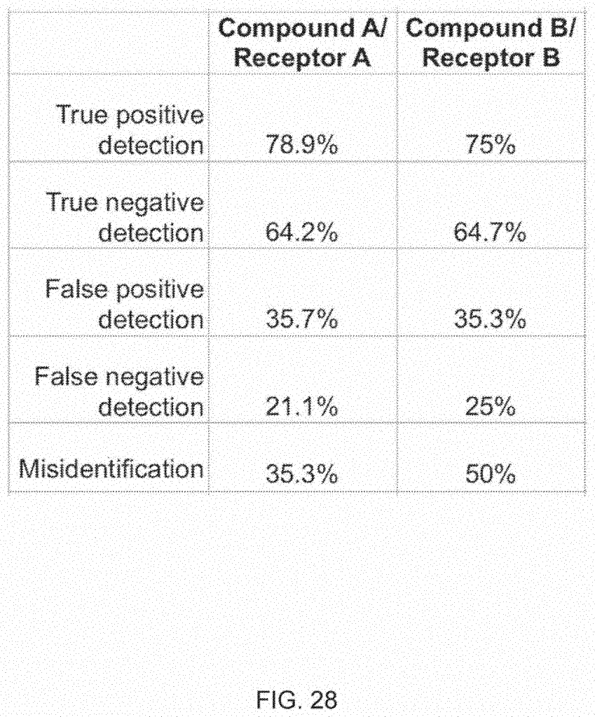

FIG. 28 provides a non-limiting example of performance test data for a prototype of the disclosed detection devices. Receptors were identified for two explosive markers—compounds A and B—and were expressed in neurons that were incorporated into the detection device prototype. The device was then exposed to air samples comprising each compound, and the activation of the neurons therein and ability of the device to discriminate between the two compounds was evaluated. The results of this trial are shown in the figure. As may be seen, the device was able to detect the presence of the two compounds with relatively high true positive detection rates (75% to 78.9%) and relatively low false positive detection rates (21.1% to 25%) and misidentification rates (35.3% to 50%).

### Example—Hospital Detection of Viral Infection

In another non-limiting example of an application for the disclosed devices and systems, a device may be positioned on a wall or other surface within a hospital. The device may be attached to the wall or surface using, for example, a structural hook. The device may comprise a plurality of neurons genetically modified to express receptors for the detection of three different types of viral particles. Ambient air from the space surrounding the device may be actively circulated through the device about every five minutes. When the ambient air contains, for example, at least about a 0.1 parts per million (ppm) concentration of at least one of the three types of viral particles, a binding event may occur between the viral particle and one or more receptors within the plurality of neurons. The binding event may generate an electrical signal (or pattern of electrical signals) that is recorded by the electrodes of a three-dimensional structured microelectrode array within the device. The electrical signal (or pattern of electrical signals) may be processed by a processor or controller of the device, and a positive indication of the presence and concentration of the viral particle may be visually and/or audibly reported by the device to a medical professional.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in any combination in practicing the invention. It is intended that the following claims define the scope of the invention and that methods and structures within the scope of these claims and their equivalents be covered thereby.

We claim:

1. A device housing comprising:
a) a shell component, wherein the shell component comprises:
i) a structure comprising a sigmoidal shape that is rotationally symmetric about a single axis;
ii) two or more air inlets positioned concentrically around the single axis;
iii) two or more air outlets positioned concentrically around the single axis; and
b) a baseplate component;
wherein the sigmoidal shape of the shell component and positions of the two or more air inlets and two or more air outlets are configured to prolong a dwell time of molecules or particles transported into an interior of the device housing by a flow of air.

2. The device housing of claim 1, wherein the baseplate component further comprises an attachment structure.

3. The device housing of claim 2, wherein the attachment structure comprises a permanent adhesive, a non-permanent adhesive, a Velcro component, a magnetic component, a hook, a wearable attachment, or any combination thereof.

4. The device housing of claim 1, wherein the shell component is an injection-molded or three-dimensional printed part.

5. The device housing of claim 1, wherein the shell component is fabricated from a polymer, a glass, a metal, a ceramic, or any combination thereof.

6. A device for detection of compounds, the device comprising:
a) a device housing;
b) a microfluidics layer comprising a fluid inlet, a fluid outlet, one or more fluid chambers, and a semipermeable membrane configured to promote gas exchange between air within the device housing and the one or more fluid chambers, wherein the one or more fluid chambers are configured to support neurons that have been genetically- engineered to express one or more odorant receptors;
c) a structured microelectrode array (MEA) comprising a plurality of electrodes configured to provide electrical stimuli to, or record electrical signals generated by, the neurons in the one or more fluid chambers.

7. The device of claim 6, wherein the device housing comprises:
a) a shell component, wherein the shell component comprises:
i) a structure comprising a sigmoidal shape that is rotationally symmetric about a single axis;
ii) two or more air inlets positioned concentrically around the single axis;
iii) two or more air outlets positioned concentrically around the single axis; and
b) a baseplate component;
wherein the sigmoidal shape of the shell component and positions of the two or more air inlets and two or more air outlets are configured to prolong a dwell time of compounds transported into an interior of the device housing by a flow of air.

8. The device of claim 6, further comprising a pre-concentrator module configured to concentrate compounds from air and maximize a dwell time of the compounds at a surface of the semipermeable membrane.

9. The device of claim 8, wherein the pre-concentrator module comprises:
a) a fan configured to draw air into the device;
b) a high efficiency particulate absorber (HEPA) filter configured to remove contaminant particles from the air drawn into the device; and
c) an air director configured to concentrate and direct the flow of air towards the surface of the semi-permeable membrane.

10. The device of claim 6, wherein the neurons have been genetically-engineered to respond to photo-stimulation, and wherein the device further comprises an light-emitting diode (LED) array configured to stimulate the neurons in the one or more fluid chambers.

11. The device of claim 6, further comprising growth medium and waste cartridges so that the device is self-contained and configured to function without maintenance for a specified period of time.

12. The device of claim 11, wherein the device is configured to function without maintenance for at least 1 week.

13. The device of claim 11, wherein the device is configured to function without maintenance for at least 1 month.

14. The device of claim 11, wherein the device is configured to function without maintenance for at least 3 months.

15. The device of claim 6, further comprising a field programmable gate array (FPGA) or processor configured to perform signal processing of electrical signals recorded by the electrodes of the MEA.

16. The device of claim 6, further comprising a field programmable gate array (FPGA) or processor configured to perform electrical stimulation of the neurons in the one or more fluid chambers using the electrodes of the MEA.

17. The device of claim 10, further comprising a field programmable gate array (FPGA) or processor configured to activate one or more LEDs of the LED array to stimulate the neurons in the one or more fluid chambers, and to perform signal processing of electrical signals recorded by the electrodes of the MEA, thereby providing a test of neuron response.

18. The device of claim 6, wherein the baseplate component comprises an attachment structure configured to attach the device to an internal or external wall, an internal or external floor, a ceiling of a room, or a roof of a building.

19. The device of claim 6, wherein the baseplate component comprises an attachment structure configured to attach the device to a bicycle, motorcycle, automobile, plane, helicopter, robot, drone, or other manned or unmanned aerial vehicle.

20. The device of claim 6, wherein the baseplate component comprises an attachment structure configured to permit the device to be worn by an animal or a human.

21-30. (canceled)
